(19) United States
(12) Patent Application Publication
(10) Pub. No.: US 2004/0062815 A1
(43) Pub. Date: Apr. 1, 2004

(54) Bdellosomes

(76) Inventors: Gerd Fricker, Staufen (DE); Rudiger Marcus Flaig, Heidelberg (DE)

Correspondence Address: MICHAEL BEST & FRIEDRICH, LLP, 100 E WISCONSIN AVENUE, MILWAUKEE, WI, 53202, US

(21) Appl. No.: 10/312,441

(22) PCT Filed: Jun. 29, 2001
(86) PCT No.: PCT/EP01/07454

(30) Foreign Application Priority Data
Jun. 29, 2000 (DE) 100 30 786.8
Oct. 30, 2000 (DE) 100 53 811.8
Apr. 11, 2001 (DE) 101 18 312.7
Apr. 17, 2001 (DE) 101 18 852.8

(51) Int. Cl. A61K 9/16; A61K 9/50

(52) U.S. Cl. 424/497

(57) ABSTRACT

The invention relates to solid particles for transportation of pharmaceutically active substances, to processes for the preparation thereof, to medicinal drugs containing said particles, and to the use of said particles for various specific indications.

The invention relates to solid particles for transportation of pharmaceutically active substances, to processes for the preparation thereof, to medicinal drugs containing said particles, and to the use of said particles for various specific indications.

A main goal of pharmaceutical research is to increase the desired effects of known active substances and to minimize systemic side effects, which is of great significance for substances exhibiting high intrinsic and thus unavoidable toxicity (eg, cytostatic drugs). This can be achieved both by reducing the total dosage required for the desired therapeutic effect and by causing accumulation of the effectors at the desired site of action, both of which possibilities may be realised by controlled, spatially specified release of effector molecules in the widest sense (proteins, peptides, nucleic acid, or low-molecular substances) in the desired target tissue. By this is meant, in particular, the specific transportation of therapeutically or diagnostically useful substances to defined biological targets (“drug delivery”, “drug targeting”), which is an important aim of present-day pharmaceutical research.

Present-day antibody technology allows for the production of highly affinic bonding partners for almost any biological structure; moreover numerous natural ligands have been characterized and cloned for cellular receptors so that it is no longer problematic to produce molecules showing high and specific affinity toward the desired targets. In many cases low-molecular ligands (eg, glycosides) are also known which can be imitated by chemical means and thus used for targeting. However these “seeker molecules” whether micromolecular or macromolecular, generally exhibit no pharmaceutically useful function, while the effectors themselves are not target-oriented. Thus it must be a main goal to overcome this discrepancy and to combine the therapeutic potential of the available effectors with the target orientation of the seeker molecules.

The most efficient attempt at achieving this goal known hither consists in the use of carrier structures of a colloidal (ie submicron) order of magnitude, to the surfaces of which appropriate target-seeking molecules can be attached. In this way there are achieved both optimal ratios of target-seeking molecules to effector molecules and maximum flexibility. Whereas only a few (<10) effector molecules can be bonded to a single antibody molecule or other ligand molecule by direct covalent attachment (which means that for a molecular weight of an antibody of approx. 150 kDa more than 90% of the mass of the conjugate is accounted for by the antibody moiety), and conjugates with low-molecular seekers usually exhibit a molar ratio of 1:1, the use of colloidal systems makes an effector to target-seeker ratio of 10³-10⁴ possible. Moreover no chemical modification of the effector is required, which is advantageous in every respect.

One efficient way of achieving this is to embed the relevant substances in colloidal carrier particles, which are linked with antibodies against, or with natural ligands for, characteristic molecular structures of the target and are at the same time protected against the immune system by an inert coating on their surface.

A much used method of colloidal packing of pharmacological agents consists in enclosing the effectors in lipid membrane-coated vesicles (liposomal artifacts or liposomes). The use of appropriate membrane components makes it possible, on the one hand, to bind the desired target-seeking molecules to the liposomal membranes, and, on the other hand, to coat the supports with anti-immunogenic material (eg, polyethylene glycol) and by this means to afford protection from non-specific removal from the bloodstream by the reticulo-endothelial system. The advantages of this system are offset by the following serious drawbacks:

The thermal and aging stability of the vesicle consisting of a single double lipid layer is limited, as is its impermeability. Theoretically, the permeability of the membranes to hydrophilic materials can be reduced, but the required modified (eg, fluorinated) lipids are not biologically acceptable. Moreover one single “direct hit” of the complementary system will suffice to cause a vesicle to run out completely.

The poor stability of the membrane vesicle in turn restricts the possibilities of varying the surface structure and thus limits the number of potential applications.

Only hydrophilic substances can be transported in the interior aqueous phase of the vesicle in adequate concentration.

Loading the liposomes is carried out (apart from a few special cases) by simply enclosing a portion of the aqueous phase and is accordingly inefficient. Typically <0.5% of the effector substance is enclosed in the vesicle. During this process the substance is exposed to considerable thermal and chemical stresses (the operating temperature must be above the critical phase transition temperature of the lipid mixture for a considerable time, and the reactive groups required for the covalent modification survive this only when the pH is very low).

Half of the membrane reactive groups required for the linkage with proteinaceous seeker molecules are, following vesicle formation, on the inside of the vesicle and are not available for linking purposes, but, when the liposomes have been assimilated, said membrane reactive groups are liberated into the organism and can lead to unforeseeable reactions.

Chemical coupling of proteins to liposomal membranes (eg, via SPDP linked directly, or through a polyethylene glycol arm, to lipids) leads to the formation of highly immunogenic structures, which have been used successfully for vaccination purposes but which must be regarded as being useless in the field of “drug targeting”, since they cause an immune reaction against the particles.

An alternative consists in the use of solid colloidal particles (“nanoparticles”). Nanoparticles are basically well known. Particles of the micrometric and submicrometric order of magnitude and comprising hydrophobic polymers can theoretically be produced by finely dispersing the polymer taken up in a non-polar solvent. Removal of the solvent causes the polymer to precipitate in the form of particles having a diameter smaller than that of a droplet; loading with hydrophobic substances (into which category most of the pharmacological agents fall) can be effected simply by adding the substance to the non-polar solvent. Following removal of the solvent, the active substance is approximately 100% associated with the polymer and remains, when the particles are present in an aqueous phase, attached to the particle matrix not by covalent bonds but by van der Waals' forces and steric entrapment, this attachment being stable for a relatively long period. An essential feature is that in this case subsequent coagulation of the hydrophobic particles (whose large surface of contact with the hydrophilic medium is energetically unfavorable) is prevented. Solutions hitherto proposed in the prior art conventionally attack this problem mostly by producing the particles in the presence of an amphiphilic substance, which mediates between the hydrophobic particle matrix and the hydrophilic medium.

Conventional nanoparticles and the preparation thereof and possibilities of surface variation are disclosed in WO 96/20698, the system here being particularly optimized for intravascular use, especially for treatment of restenosis. The Patent Application describes polymolecular nanoparticles of natural or synthetic polycondensates and contains a predominantly general description of a property-modifying surface coating of natural or synthetic macromolecules.

The the following documents may also be mentioned by way of example.

DE 198 109 65 A1 describes polymolecular nanoparticles of a polyelectrolyte complex of polycations and polyanions which is treated with a crosslinking agent.

U.S. Pat. No. 6,117,454 describes, in particular, polymolecular nanoparticles which are made suitable for penetration of the blood-brain barrier by a coating of fatty acid derivatives. In the case of these intrinsically amphiphilic ligands it is necessary to consider, on the one hand, that highly affinic ligands, which are in the widest sense active against biological structures (eg, proteins), are generally not available on such a scale that they could be suitable, even when appropriate physical properties are given (which does to apply to, say, antibodies), for direct surface coating (for which purpose quantities comparable with the particle matrix would be necessary), and also, on the other hand, even where this is possible, it is strongly advised not to attach such masses in a highly affinic manner to molecules binding biological targets, of which it can be expected that they will partially release themselves from the particles and, moreover, will bind to their targets.

U.S. Pat. No. 5,840,674 describes complexes of active substances and microparticles, which complexes are permanently covalently attached via a “linker”, particularly for use against microorganisms.

U.S. Pat. No. 5,641,515 describes polymolecular nanoparticles of polycyanoacrylate containing insulin, which effect controlled release of the coordinated insulin.

DE 198 395 15 A1 describes colloidal associates of polymer and active substance containing a property-optimized branched polyol ester particularly for use on mucous tissues. This is achieved by esterifying a polymeric polyalcohol, particularly polyvinyl alcohol, with, for example, polycondensing hydroxycarboxylic acids so that, starting from the polyol backbone, polycondensed side chains of different lengths and a free terminal OH group are formed with the intention of modifying the properties of the polyol ester. The fact that the side chains of the particles produced according to DE 196 395 15 A1 terminate exclusively in free OH groups is very disadvantageous, since OH groups cannot be caused to react selectively in an aqueous or alcoholic environment with the result further surface modification, for example, with “seeker” molecules, is difficult or almost impossible. Furthermore, the particle synthesis described in DE 198 395 15 necessarily involves a dispersion step in an aqueous phase, so that particles produced by this process cannot be modified further. In particular, however, the particles of DE 198 395 15 also do not have an adequately controlled clearly defined structure, since no molecules are added, by means of which the length of the side chains could be controlled in a defined manner by chain terminating reactions, and also no groups conducive to further modification are introduced into the surface of the molecule. In addition, the manufacturing process of the polymer is, inter alia on account of the terminal OH groups, much more elaborate and (on account of the influence of numerous process parameters) less robust and also leads to a polymer of less-defined structure, which is not capable of automatically forming monomolecular particles but is converted “by controlled precipitation in colloidal form”. The resultant “colloidal supramolecular assemblies (associates)” do not have, on account of the absence of defined terminators, a surface layer which is chemically different from the rest of the particle although covalently linked thereto, and by means of which said defined surface modification could be carried out. Taken as a whole, the particles described in DE 198 395 15 are consequently unsuitable for use in the field of “drug targeting”, particularly by surface modification in the sense of this invention, and can only be used as “sustained release” formulations.

Thus, in all, the prior art only inadequately solves the problem of achieving precise dosage of the active substance at the desired site of action and of attaining stability of the nanoparticles from the time of administration to their arrival at the site of action, ie the problem of “drug targeting”. A central problem relates to the surface modification of nanoparticles. As mentioned above, conventional solid nanoparticle systems constitute, unlike liposomal artifacts, an energetically unfavorable system by reason of their extremely high specific interphase between the hydrophobic particle matrix and the hydrophilic medium, which system is unstable in the absence of stabilizing agents. Agglomeration of the particles minimizes the energetically unfavorable surface of contact, but this leads to precipitation of the hydrophobic particulate substance.

For this reason, conventional nanoparticles require a coating of, for example, amphiphilic molecules, which lower the interfacial energy and in this way stabilize the particles. This coating covers the particle matrix completely and allows no access of modifying agents thereto. On the other hand, modification effected by just such amphiphilic molecules leads, however, to a drastic change in the physical properties and thus to destabilization of the coating. For this reason, it is scarcely possibly to modify conventional nanoparticles such that binding of ligands and thus the use thereof in the field of drug targeting is possible, which, as already stated, also applies to DE 198 395 15.

In addition to overcoming the said drawbacks of the prior art, it is an object of the invention to provide nanoparticles

(a) which can transport a large spectrum of active substances by “steric entrapment”,

(b) which can undergo stable surface-modification in a simple manner such that they

(c) exhibit defined and suitable chemical groups on their surface or can be readily synthesized with such groups, and

(d) show a defined size and, in particular, a defined shape and surface of the molecule

and, in particular, are capable of effecting specific release at the site of action.

This object is achieved by means of solid particles for transportation of hydrophobic pharmaceutical active substances which contain

a) an unbranched or at most triple-branched molecular backbone of a polymer composed of monomers and having at least one bonding group (x) on each monomer, there being attached to each of the bonding groups, (x) via a covalent (x)-(x′) bond,

b) polycondensed molecular side chains composed of chain-forming monomers having in each case at least one bonding group (y) and at least one bonding group (x′) or composed of different chain-forming monomers, of which one monomer has at least two bonding groups (y) and a different monomer has at least two bonding groups (x′), or composed of different chain-forming monomers, of which one monomer has at least two bonding groups (y), a different monomer has at least two bonding groups (x′) and another monomer has at least one bonding group (y) and at least one bonding group (x′), where (x′) is a group capable of forming a covalent bond with (x) and also a covalent bond with (y), whilst at the end of each molecular side chain there is attached, via a (y)-(y′) covalent bond

c) a side-chain terminator having at least one bonding group (y′), no bonding group (y), and at least one free group (z) optionally provided with a protective group, in which

the molar ratio of the monomers in the molecular side chains (b) to the side-chain terminators (c) is >>1,

group y≠group z and group x′≠group z,

the molar ratio of the monomers in the molecular backbone to the side-chain terminators (c) is approximately equimolar,

x, x′, y and y′ are independently selected from the group comprising OH, SH, COOH and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide bond NH—C(O)—), SH/COOH (thioester bond —S—C(O)—), COOH/OH (ester bond —O—C(O)—), COOH/NH₂ (amide bond —NH—C(O)—), and COOH/SH (thioester bond S—C(O)—) and

z is selected from the group comprising CH₃, OH, SH, COOH, and NH₂, and also the vinyl or epoxide group, optionally protected by a suitable protective group.

Protective groups for certain functional radicals and the removal thereof are well known to the person skilled in the art. Possible protective groups are, for example, FMOC for the protection of an amino function, in which case the removal thereof is carried out by treatment with catalytic amounts of piperidine.

The particles of the invention are particularly suitable forms, by means of which there is achieved increased action and minimization of the side effects by the controlled and/or spatially specific release of the effector molecules. Generally the particles involved in the invention constitute preferably solid, colloidal and/or lipid-free particle systems.

A special advantage of the groups of the invention is that, due to the presence of functional groups not otherwise present in the polymer particle, particularly amino groups, the surface layer is such that

the inclusion of these functional groups causes the particles to be encased in a polar zone whose charge hinders flocculation and thus stabilizes the suspension of particles, and

there are provided functional groups (particularly, but not necessarily exclusively, amino groups) which are suitable for adding surface modifications following formation of the particles loaded with the substance of interest.

Furthermore, the functional groups the surface can be deliberately selected, depending on the task concerned, via the side-chain terminators without causing any problems, and the manufacturing process for the compounds of the invention is simple and robust.

Besides, the size, shape, and surface of the molecule can be clearly defined in a controlled manner, since the choice of the molar ratios determines the structure of the resultant molecule, whilst reaction parameters such as time, temperature, pressure, etc. have no significant influence under standard conditions.

Furthermore, due to the polymeric side chains, the particles are highly capable of non-covalently binding, by steric entrapment, and thus of transporting, large amounts of a wide variety of active substances having various chemical properties.

The invention also relates to solid particles for transportation of amphiphilic or lipophilic active substances or hydrophobic resorption esters of hydrophilic active substances, containing a molecule of a branched polycondensate, the polycondensate consisting of a backbone of a polyfunctional, preferably unbranched or not more than triple-branched, macromolecule, preferably polyvinyl alcohol, whose functional groups (in the case of polyvinyl alcohol, the OH groups) are linked by a covalent bond to secondary, preferably likewise unbranched or not more than triple-branched, polycondensate chains, which comprise secondary polycondensate chains of linked bifunctional organic monomers (which monomers can be heterobifunctional molecules or derivatives thereof or an equimolar mixture of two homobifunctional molecules or derivatives thereof), preferably of esterified hydroxycarboxylic acids or a combination of diols and dicarboxylic acids in a molar ratio of 1:1, which monomers are designated below as side chains, the end of each of these chains consisting of a molecule which is monofunctional as regards the condensation reaction, preferably a non-hydroxycarboxylic acid. A special feature is that this molecule, which is monofunctional as regards the condensation reaction, is provided with a second functional group not otherwise present in the molecule and protected during the condensation reaction by a protective group, which second functional group can be eliminated on conclusion of the condensation reaction in a second step so that after the second reaction step the chains terminate in specific functional groups.

FIG. 2a shows diagrammatically, by way of example, the formation and structure of a general polycondensate consisting of an unbranched, multifunctional backbone, a heterobifunctional side-chain monomer, and a terminator appropriate to said combination. Of course, it is possible to combine more than one type of monomer and/or terminator in a single synthesis reaction. Such a polycondensate having an unbranched backbone and no or only a few branched side chains is referred to below as ktenate by reason of its shape (Greek kteis=comb).

In this case formation of the side chains can involve the use of the original monomers which split off water or other small molecules in the condensation reaction, or the corresponding derivatives thereof resulting from elimination of small molecules eg, anhydrides, lactones etc., or other derivatives. The use of “preformed” oligomers having freely available functional groups for the condensation reaction (eg, oligopeptides) alone or in arbitrary combinations with homobifunctional or heterobifunctional monomers is likewise possible. Such pre-formed building blocks are subsumed below under the term “monomers”.

The following are examples of some of the substances and combinations which may be used within the scope of the invention. The lists are not claimed to be fully comprehensive.

| NAME | BASIC STRUCTURE | FUNCTIONAL GROUP |
|---|---|---|
| Backbone | | |
| Polyvinyl alcohol | H2(CH2—CHOH)n | —OH |
| Polyacrylic acid | H2(CH2—CHCOOH)n | —COOH |
| Polyvinyl amine | H2(CH2—CHNH₂)n | —NH₂ |
| Polysaccharides | various | —OH |
| Polyamino acids | various | various |
| NAME | BASIC STRUCTURE | SUITABLE BACKBONE |
| Side-chain monomers | | |
| Hydroycarboxylic acids | HOOC—X—OH | Polyvinyl alcohol, |
| | | polyacrylate, |
| | | polysaccharides, |
| | | polyamines |
| Diols +dicarboxylic | HO—X—OH + HOOC—Y—COOH | Polyvinyl alcohol, |
| acids | | polyacrylate, |
| | | polysaccharides, |
| | | polyamines |
| Amino acids | HOOC—X—NH2 | Polyamines, polyacrylate |
| Diamines +dicarboxylic | H2N—X—NH₂ + HOOC—Y— | Polyamines, polyacrylate |
| acids | COOH | |

| | BASIC | DEPROTECTED | |
|---|---|---|---|
| NAME | STRUCTURE | END GROUP | EXAMPLE |
| Terminators | | | |
| N-protected amino | HOOC—X—NH-Ω | —NH₂ | N-FMOC—alanine |
| acids | | | |
| COOH-protected | H2N—X—COO-Ω | —COOH | Ω-alanine |
| amino acids | | | |
| N-protected | HO—X—NH-Ω | —NH₂ | N-FMOC-colamine |
| aminoalcohols | | | |
| O-protected | H2N—X—O-Ω | —OH | O-Ω-colamine |
| aminoalcohols | | | |
| S-protected thiols | HO—X—S-Ω | —SH | S-Ω-β-mercapto- |
| | | | ethanol |
| S-protected thiollic | HOOC—X—S-Ω | —SH | S-Ω-thioglycolic acid |
| acids | | | |

(X and Y are any one molecular body Ω = protective group)

The selection of the terminators depends on the following factors:

the desired exposed group(s) (should be provided with a suitable protective group prior to synthesis to prevent involvement in the polycondensation process);

the side-chain monomer(s)

the backbone molecule (defines orientation of the side chains through its functional groups).

These side chains are generally designated as telo-terminator-poly-monomer (ie teloalanylpolylactide or similar), thus the entire molecule as backbone-_{mol.wt.}-telo{free group}terminator-poly-monomer_{average side-chain-weight}ate, eg, polyvinyl 200,000-telo{amino}alanylpolylactide₅₀₀₀ate, polyacryl50,000-telo{amino,sulfhydro}cysteylpoly (glycol:adipin)8000ate (or similar).

An overview of a selection of possibilities is listed in the following table, in which trivial names are also proposed for some of the most interesting basic structures:

| | Side chain | | Dominant | Deprotected | |
|---|---|---|---|---|---|
| Backbone | material | Terminators | bond | surface group | Trivial name |
| Polyvinyl | Hydroxycar- | N-protected amino | Ester | —NH₂ | Regular |
| alcohol or | boxylic acids or | acids | | | ktenate |
| some other | dicarboxylic acids | N-protected and | | —NH₂, —COOH | Acid regular |
| hydroxy | and diols 1:1 | sec-COOH— | | | ktenate |
| compound | | protected amino | | | |
| | | dioic acids | | | |
| | | N-protected diamino | | —NH₂, —NH₃+ | Basic regular |
| | | acids | | | ktenate |
| | Thiohydroxy | N-protected amino | Ester, | —NH₂ | Oxidatively |
| | carboxylic acids | acids | disulfide | | cross-linked |
| | | | | | ktenate |
| | Hydroxy | S-protected | Ester | —SH | Thioknetate |
| | carboxylic acids | thiocarboxylic acids | | | |
| | or dicarboxylic | | | | |
| | acids and diols | | | | |
| | 1:1 | | | | |
| Polyacrylic | Hydroxycar- | N-protected amino | Ester | —NH₂ | Inverse ktenate |
| acid and | boxylic acids or | alcohols | | | |
| other | dicarboxylic acids | | | | |
| macromolecular | and diols 1:1 | | | | |
| polyacids | Amino acids or | N-protected amino | Peptide | | Inverse |
| | dicarboxylic acids | acids | | | amidoktenate |
| | and diamines 1:1 | | | | |
| | Cysteine | | Peptide, | | Cross-linkable |
| | | | disulfide | | inverse |
| | | | | | amidoktenate |
| | Hydroxycar- | S-protected | Ester | —SH | Inverse |
| | boxylic acids or | thioalcohols | | | thioktenate |
| | dicarboxylic acids | | | | |
| | and diols 1:1 | | | | |
| Macromolecular | Amino acids or | S-protected | Peptide | —SH | Thioamide |
| polyamines | dicarboxylic acids | thioamines | | | ktenate |
| | and diamines 1:1 | Partially protected | | —NH₂ | Amidoktenate |
| | | diamines | | | |
| | | COOH-protected | | —SH, —COOH | Acid |
| | | and S-protected | | | amidoktenate |
| | | cysteine | | | |

All of these possibilities are subject matter of this invention irrespective of the type and orientation of the bonding structure dominating the particle matrix, since they all result in the same elongated particle shape and structure comprising a hydrophobic core and a functionalized, hydrophilic outer layer, cohering by covalent bonds. The structure is designated below as “bdellosome” (Greek bdella=leech) and satisfies the aforementioned criteria.

Thus the following description relates exemplarily only to the synthesis and application of regular lactalanine ktenate (exact designation according to above nomenclature: polyvinylteloaminoalanylpolylactidate), as the other basic structures give particles which are not substantially different.

The polymers described in the list differ in suitability depending on their objective; thus, for example, amino-containing, but thiol-free seeker molecules can, with appropriate variation of the protocol (first coupling of seeker and linker, then reaction of the particle with the seeker/linker complex), be attached, by way of the conventional NHS-ester-PEG-vinylsulfone linker (here in reversed orientation), to particles of thioktenate, inverse thioktenate, or thioamidoktenate.

The particles of the invention very preferably contain non-covalently bonded, hydrophobic or hydrophobed pharmaceutically active substance.

By hydrophobed active substances we mean active substances which were originally more hydrophilic but have been rendered more hydrophobic by chemical modification. As an example there may be mentioned hydrophobic resorption esters of hydrophilic active substances.

Another preferred subject of the invention, which also achieves the above object and exhibits the said preferred properties, comprises monomolecular solid particles for transportation of hydrophobic or hydrophobed active substances, produced by a process in which an unbranched or at most triple-branched polymer backbone composed of monomers and having at least one bonding group (x) on each monomer is brought into contact with

(a) chain-forming side-chain monomers each having at least one bonding group (y) and at least one bonding group (x′), where (x′) is a group capable of forming a covalent bond both with (x) and with (y),

(b) a mixture of chain-forming side-chain monomers, of which at least one monomer has at least two bonding groups (y) and at least one other monomer has

at least two bonding groups (x′), where (x′) is a group capable of forming a covalent bond both with (x) and with (y), or

(c) a mixture of chain-forming side-chain monomers, of which at least one monomer has at least two bonding groups (y), at least one other monomer has at least two bonding groups (x′) and at least one further monomer has at least one bonding group (y) and at least one bonding group (x′), where (x′) is a group capable of forming a covalent bond both with (x) and with (y),

and at least one side-chain terminator having at least one bonding group (y′) but not having any bonding group (y) and having at least one free group (z) optionally provided with a protective group, where (y′) is a group capable of forming a covalent bond with (y),

under conditions which permit linkage of the monomers or monomer mixtures in the side chains to the polymer backbone and to the chain terminators

and also permit polycondensation of the monomers or monomer mixtures in the side chains, in which

the molar ratio of the monomers in the molecular side chains (b) to the side-chain terminators (c) is >>1,

group y≠group z and group x′≠group z,

the molar ratio of the monomers in the molecular backbone to the side-chain terminators (c) is approximately equimolar,

x, x′, y and y′ are independently selected from the group comprising OH, SH, COOH, and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide bond NH—C(O)—), SH/COOH (thioester bond —S—C(O)—), COOH/OH (ester bond —O—C(O)—), COOH/NH₂ (amide bond —NH—C(O)—) or COOH/SH (thioester bond —S—C(O)—,

z is selected from the group comprising CH₃, OH, SH, COOH, NH₂, and the vinyl or epoxide group optionally protected by a suitable protective group and

the side-chain monomers can be used as pure monomers or derivatives such as inner anhydrides or lactones provided they can still form chains with themselves and/or with other side-chain monomers.

Another preferred object the invention, which achieves the above object and has the said preferred properties, comprises monomolecular solid particles for transportation of hydrophobic or hydrophobed active substances, produced by a process, in which an unbranched or at most triple-branched polymer backbone composed of monomers each having at least one bonding group (x) on each monomer is brought into contact with

polycondensed molecular side chains composed of monomers each having at least one bonding group (y) and at least one bonding group (x′), or composed of different monomers, of which one monomer has at least two bonding groups (y) and a different monomer has at least two bonding groups (x′), or composed of different monomers, of which one monomer has at least two bonding groups (y), a different monomer has at least two bonding groups (x′) and another monomer has at least one bonding group (y) and at least one bonding group (x′), where (x′) is a group capable of forming a covalent bond both with (x) and with (y), and at the end of each molecular side chain there is covalently bonded thereto, over a (y)-(y′) bond, side-chain terminators having at least one bonding group (y′), no bonding group (y), and at least one free group (z), optionally provided with a protective group, where (y′) is a group capable of forming a covalent bond with (y),

under conditions which permit linkage between the polycondensed molecular side chains and the polymer, in which

the molar ratio of the monomers in the molecular side chains (b) to the side-chain terminators (c) is >>1,

group y≠group z and group x′≠group z,

the molar ratio of the monomers in the molecular backbone to the side-chain terminators (c) is approximately equimolar,

x, x′, y and y′ are independently selected from the group comprising OH, SH, COOH, and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide bond NH—C(O)—), SH/COOH (thioester bond —S—C(O)—), COOH/OH (ester bond —O—C(O)—), COOH/NH₂ (amide bond —NH—C(O)—) or COOH/SH (thioester bond —S—C(O)— and

z is selected from the group comprising CH₃, OH, SH, COOH, NH₂, and the vinyl or epoxide group, optionally protected by a protective group.

For example, the free OH groups in the polyvinylalcohol backbone are esterified with the polycondensate-forming hydroxycarboxylic acid whilst the non-hydroxycarboxylic acids serve as terminators for these side chains. The function of the non-hydroxycarboxylic acid is here analogous to that of free-radical scavengers used to fix the average chain length in free-radical polymerization reactions.

It is preferred that contacting takes place in an anhydrous organic solvent, preferably pyridine, and it is an advantageous and preferred embodiment of the process to effect contacting in the presence of thionyl chloride and/or, if necessary, to eliminate protective groups, for example following the reaction with thionyl chloride. Protective groups for certain functional radicals and the removal thereof are well known to the person skilled in the art. A possible protective group could be, for example, FMOC for the protection of an amino function, the removal thereof being effected by treatment with catalytic amounts of piperidine.

Usually a washing step follows either after the elimination of protective groups or—if this is not necessary—directly, after the reaction with thionyl chloride, for example, and said washing is preferably effected with dichloromethane, but can, of course, be carried out using other anhydrous—usually non-polar—organic solvents.

Some preferred embodiments and advantageous effects which can be achieved with the present invention are described below. The statements made herein refer to all products and processes of the invention, as described herein.

The preferred particles of the present invention are monomolecular solid particles. These comprise a bdellosome molecule of the invention. It has been found, surprisingly, that monomolecular particles for the transportation of active agent can be formed from the bdellosomes described herein. These particles are characterized by an average particle size which is smaller than that of classical liposomal artifacts. Preferred monomolecular particles of the invention have a length, diameter, and volume as stated in the application. Both the size and the shape of the particle can be controlled by suitable modification of the polymer skeleton. A long polymer backbone having, by comparison, shorter side chains gives oblong particles, whereas a short polymer backbone having long side chains gives particles which are more spherical. However, the bdellosomes usually and preferably form oblong particles, and particles having a length of ca 2 μm and a diameter of as small as 5 nm can be produced, compared with the usually spherical liposomal artifacts. Both the shape and the small size compared with classical liposomal artifacts present great advantages for targeted administration of active substances, since the particles of the invention make it possible to use routes to cells which are not possible with the classical liposomal artifacts by reason of their size and/or shape.

The molecular backbone of polymer composed of monomers can be a backbone of only one type of monomer (homopolymer) or a backbone of more than one type of monomer (copolymer). Preferred polymers composed of only one monomer for the backbone are polyvinyl alcohol, polyvinyl amine, or polyacrylic acid. The copolymers composed of more than one monomer are preferably those comprising from 2 to 5, more preferably 2, different monomers. These monomers can be selected from the monomers mentioned in the description as being suitable for the backbone. Preference is again given to monomers for polyvinyl alcohol, polyvinyl amine, or polyacrylic acid.

The molecular backbone preferably comprises, on average, from 500 to 20,000, more preferably from 1,000 to 10,000, and most preferably from 2,000 to 7,000, monomer units.

The monomer units for the polymer backbone contain in each case at least one bonding group x, preferably from 1 to 4 bonding groups x, and more preferably 1 or 2 bonding groups x.

The molecular side chains comprise, on average, preferably from 10 to 10,000, more preferably from 50 to 5000, and most preferably from 80 to 2000, monomer units. The ratio of monomer units in the side chain to the side-chain terminator is usually very much greater than one. Preference is given to the ranges indicated by the ranges given above for the number at monomer units in the molecular side chain.

The molar ratio of monomers in the molecular backbone to the side-chain terminators is usually approximately equimolar, preferably from 1:0.8 to 0.8:1, more preferably from 1:0.9 to 0.9:1, and most preferably equimolar. These statements refer to unbranched molecular side chains. However, the molecular side chains can be branched, a low degree of branching, if any, being preferred, ie preferably from 1 to 4 branches per side chain, and more preferably 1 or 2 branches per side chain. When such branching is present, the molar ratio of monomers in the molecular backbone to the side-chain terminators changes accordingly.

The linkers, preferably linkers of polyalkylene glycols, more preferably polyethylene glycol, usually have a molar mass in a range as is usual for these molecules, for example, as given in the examples, but preferably in the range of from 500 to 10,000, and more preferably from 1,500 to 5,000.

The side-chain terminators can exhibit free groups (z). The side-chain terminators may be selected such that a variable portion of side chains exhibits a free group (z). Thus from 100% to 0% of the side chains can have a free group (z), depending on how much further modification is desired. Suitable contents of free groups (z) are, for example, from 1 to 10%, from 1 to 25%, or alternatively from 50 to 99%, from 75 to 95%, and from 80to 90%.

Suitable selection of the components within the aforementioned ranges can control the surprising effects achieved by the present invention.

The monomolecular particles of the invention can be produced in a simple manner in good yields from the polymers described herein and loaded with active substances. It is usually sufficient to effect simple dissolution in a suitable solvent containing the desired active substance. Surprisingly, large portions of the polymers can be converted to monomolecular particles, loaded with the active substance, without mechanical treatment, which is not detrimental, however. In the present invention, conversions of more than 50% can be achieved, preferably more than 80%, more preferably more than 90%, and most preferably more than 95%, by weight. This produces particles in the desired submicron range.

The monomolecular particles preferred within the scope of the invention are especially suitable for transporting molecules of active substance. For this reason, the present invention also claims the use of said particles in the production of medicinal drugs and for the administration of active substances. The invention also relates to a method of administrating active substances, in which particles of the invention are used as vehicles for active substances. Here again, the preferred ranges described in the present application are applicable.

Alternative methods of producing the ktenate, eg, by solid-phase synthesis according to Merrifield, are likewise an object of the present invention, provided the basic structure of the molecules produced complies with the specified definitions of a ktenate.

In use, the resulting particles are loaded with the hydrophobic or hydrophobed pharmaceutically active substance to be transported. Thus another process step follows, in which the product, together with the hydrophobic or hydrophobed pharmaceutically active substance to be transported, is dissolved in an anhydrous organic solvent, after which the solution is incubated for a period of time, preferably overnight, and preferably at room temperature, and the solution is then saturated with water and the water-saturated solution is then dissolved in a large volume of water, followed, optionally, by mechanical treatment (preferably not using supersonics), after which the particles are cleaned if necessary and then isolated.

However, such treatment is not usually necessary, and it is preferred, within the scope of this invention, that no mechanical treatment follows dissolution of the water-saturated solution in a large volume of water.

Final cleaning (and isolation)—if at all necessary at this stage of the process—is preferably effected by dialysis.

As regards the selection of solvent for the step of loading with active substance, it is preferred that the anhydrous organic solvent dissolves in water in a ratio of solvent to water between 1:10 and 1:50, preferably between 1:20 and 1:40, and more preferably between 1:20 and 1:30, and/or is preferably selected from:

dichloromethane or benzyl alcohol, preferably benzyl alcohol.

However, the selection need not be restricted thereto, provided a specific degree of water solubility is given.

It is particularly preferred that the polymer backbone of the particles of the invention be unbranched or branched only once, but is preferably unbranched.

Particularly preferred embodiments of the particles of the invention are subject to the following individual conditions, or to some or all of the following conditions, namely:

the monomers in the side chain each contain not more than two groups (y) and not more than two groups (x′) and/or

group (y) in the monomers in the side chain is the same as group (x) in the polymer backbone and/or

group (x′) in the monomers in the side chain is the same as group (y′) in the side-chain terminators and/or

group (z) is selected from the “free groups” OH, SH, COOH, NH₂, and the vinyl or epoxide group optionally protected by a protective group,

the monomers in the side chain each have not more than from 2 to 10 carbon atoms, preferably from 2 to 6 carbon atoms, and more preferably from 2 to 4 carbon atoms and/or

the monomers in the side chain which contain both group (y) and group (x′), have either only 1 group (y) and 1 or 2, preferably 1, groups (x′) or only 1 group (x′) and 1 or 2, preferably 1, groups (y) and/or

each of the monomers in the side chains are, except for the side-chain terminators, identical to 1 group (y) and 1 group (x′) or the monomers in the side chains are, except for the side-chain terminators, identical to, in monotonously alternating relationship, a monomer having 2 groups (x′) alternating with a monomer having 2 groups (y).

The expression “free groups” defining group (z) as being selected from the group comprising OH, SH, COOH, NH₂, and the vinyl or epoxide group, optionally protected by a protective group, is a fixed definition for the purposes of the present invention.

In another preferred embodiment of the particles of the invention, the polymer backbone is selected from the group comprising

polyvinyl alcohol, polyacrylic acid, polyvinyl amine, polysaccharide, and polyamino acid,

preferably polyvinyl alcohol and polyacrylic acid,

and more preferably polyvinyl alcohol.

In another preferred embodiment of the particles of the invention, the monomers in the side chain are selected from the group comprising

hydroxycarboxylic acids, amino acids, a combination of diamines and dicarboxylic acids or a combination of diols and dicarboxylic acids, or derivatives thereof,

preferably hydroxycarboxylic acids or a combination of diols and dicarboxylic acids, or derivatives thereof,

particularly hydroxycarboxylic acids such as lactic acid, glycolic acid, tartaric acid, citric acid, or derivatives thereof.

In another preferred embodiment of the particles of the invention, the side-chain terminators are selected from the group comprising

unprotected amino acids, N-protected amino acids, COOH-protected amino acids, unprotected amino alcohols, N-protected amino alcohols, O-protected amino alcohols, unprotected thiols, O-protected thiols, S-protected thiols, or unprotected thiolic acids, S-protected thiolic acids, COOH-protected thiolic acids, unprotected thioamines, S-protected thioamines, or N-protected thioamines, preferably

unprotected amino acids, N-protected amino acids, unprotected amino alcohols, N-protected amino alcohols, unprotected thiols, S-protected thiols, or unprotected thiolic acids, and S-protected thiolic acids,

more preferably

unprotected amino acids, such as alanine, N-protected amino acids, such as, N-FMOC-β-alanine, unprotected thiolic acids, or S-protected thiolic acids.

The expression “A-protected” means, for the purposes of the invention, that a functional group “A” of the relevant molecule is provided with a protective group of arbitrary nature capable of being removed following the condensation reaction.

In a very particularly preferred embodiment of the particles of the invention, the polymer backbone, the monomers in the side chain or derivatives thereof and the side-chain terminators or derivatives thereof are selected from the group comprising one of the following combinations:

| Comb. | Polymeric | Monomer(s) in side-chain | Side-chain terminator |
|---|---|---|---|
| No. | backbone | or derivative(s) thereof | or derivative thereof |
| 1 | Polyvinyl alcohol | Hydroxycarboxylic acid | Unprotected amino acid |
| 2 | Polyvinyl alcohol | Hydroxycarboxylic acid | N-protected amino acid |
| 3 | Polyvinyl alcohol | Hydroxycarboxylic acids | Unprotected thiolic acid |
| 4 | Polyvinyl alcohol | Hydroxycarboxylic acids | S-protected thiolic acid |
| 5 | Polyvinyl alcohol | Combination of diol and dicarboxylic acid | Unprotected amino acid |
| 6 | Polyvinyl alcohol | Combination of diol and dicarboxylic acid | N-protected amino acid |
| 7 | Polyvinyl alcohol | Combination of diol and dicarboxylic acid | Unprotected thiolic acid |
| 8 | Polyvinyl alcohol | Combination of diol and dicarboxylic acid | S-protected thiolic acid |
| 9 | Polyvinyl alcohol | Amino acid | Unprotected thiolic acid |
| 10 | Polyvinyl alcohol | Amino acid | S-protected thiolic acid |
| 11 | Polyvinyl alcohol | Combination of diamine and dicarboxylic acid | Unprotected thiolic acid |
| 12 | Polyvinyl alcohol | Combination of diamine and dicarboxylic acid | S-protected thiolic acid |
| 13 | Polyacrylic acid | Hydroxycarboxylic acid | Unprotected amino alcohol |
| 14 | Polyacrylic acid | Hydroxycarboxylic acid | N-protected amino alcohol |
| 15 | Polyacrylic acid | Hydroxycarboxylic acid | Unprotected thiol |
| 16 | Polyacrylic acid | Hydroxycarboxylic acid | S-protected thiol |
| 17 | Polyacrylic acid | Combination of diol and dicarboxylic acid | Un rotected amino alcohol |
| 18 | Polyacrylic acid | Combination of diol and dicarboxylic acid | N-protected amino alcohol |
| 19 | Polyacrylic acid | Combination of diol and dicarboxylic acid | Unprotected thiol |
| 20 | Polyacrylic acid | Combination of diol and dicarboxylic acid | S-protected thiol |
| 21 | Polyacrylic acid | Amino acid | Unprotected amino alcohol |
| 22 | Polyacrylic acid | Amino acid | O-protected amino alcohol |
| 23 | Polyacrylic acid | Amino acid | Unprotected thioamine |
| 24 | Polyacrylic acid | Amino acid | S-protected thioamine |
| 25 | Polyacrylic acid | Combination of diamine and dicarboxylic acid | Unprotected amino alcohol |
| 26 | Polyacrylic acid | Combination of diamine and dicarboxylic acid | O-protected amino alcohol |
| 27 | Polyacrylic acid | Combination of diamine and dicarboxylic acid | Unprotected thioamine |
| 28 | Polyacrylic acid | Combination of diamine and dicarboxylic acid | S-protected thioamine |
| 29 | Polyvinyl amine | Hydroxycarboxylic acids | Unprotected amino acid |
| 30 | Polyvinyl amine | Hydroxycarboxylic acids | N-protected amino acid |
| 31 | Polyvinyl amine | Hydroxycarboxylic acids | Unprotected thiolic acid |
| 32 | Polyvinyl amine | Hydroxycarboxylic acids | S-protected thiolic acid |
| 33 | Polyvinyl amine | Combination of diol and dicarboxylic acid | Unprotected amino acid |
| 34 | Polyvinyl amine | Combination of diol and dicarboxylic acid | N-protected amino acid |
| 35 | Polyvinyl amine | Combination of diol and dicarboxylic acid | Unprotected thiolic acid |
| 36 | Polyvinyl amine | Combination of diol and dicarboxylic acid | S-protected thiolic acid |
| 37 | Polyvinyl amine | Amino acid | Unprotected thiolic acid |
| 38 | Polyvinyl amine | Amino acid | S-protected thiolic acid |
| 39 | Polyvinyl amine | Combination of diamine and dicarboxylic acid | Unprotected thiolic acid |
| 40 | Polyvinyl amine | Combination of diamine and dicarboxylic acid | S-protected thiolic acid |
| 41 | Polysaccharide | Hydroxycarboxylic acid | Unprotected amino acid |
| 42 | Polysaccharide | Hydroxycarboxylic acid | N-protected amino acid |
| 43 | Polysaccharide | Hydroxycarboxylic acids | Unprotected thiolic acid |
| 44 | Polysaccharide | Hydroxycarboxylic acids | S-protected thiolic acid |
| 45 | Polysaccharide | Combination of diol and dicarboxylic acid | Unprotected amino acid |
| 46 | Polysaccharide | Combination of diol and dicarboxylic acid | N-protected amino acid |
| 47 | Polysaccharide | Combination of diol and dicarboxylic acid | Unprotected thiolic acid |
| 48 | Polysaccharide | Combination of diol and dicarboxylic acid | S-protected thiolic acid |
| 49 | Polysaccharide | Amino acid | Unprotected thiolic acid |
| 50 | Polysaccharide | Amino acid | S-protected thiolic acid |
| 51 | Polysaccharide | Combination of diamine and dicarboxylic acid | Unprotected thiolic acid |
| 52 | Polysaccharide | Combination of diamine and dicarboxylic acid | S-protected thiolic acid |
| 53 | Polycystein | Hydroxycarboxylic acids | Unprotected amino acid |
| 54 | Polycystein | Hydroxycarboxylic acids | N-protected amino acid |
| 55 | Polycystein | Hydroxycarboxylic acids | Unprotected thiolic acid |
| 56 | Polycystein | Hydroxycarboxylic acids | 5-protected thiolic acid |
| 57 | Polycystein | Combination of diol and dicarboxylic acid | Unprotected amino acid |
| 58 | Polycystein | Combination of diol and dicarboxylic acid | N-protected amino acid |
| 59 | Polycystein | Combination of diol and dicarboxylic acid | Unprotected thiolic acid |
| 60 | Polycystein | Combination of diol and dicarboxylic acid | S-protected thiolic acid |
| 61 | Poiycystein | Amino acid | Unprotected thiolic acid |
| 62 | Polycystein | Amino acid | S-protected thiolic acid |
| 63 | Polycystein | Combination of diol and dicarboxylic acid | Unprotected thiolic acid |
| 64 | Polycystein | Combination of diol and dicarboxylic acid | S-protected thiolic acid |
| 65 | Polyserin | Hydroxycarboxylic acid | Unprotected amino acid |
| 66 | Polyserin | Hydroxycarboxylic acid | N-protected amino acid |
| 67 | Polyserin | Hydroxycarboxylic acids | Unprotected thiolic acid |
| 68 | Polyserin | Hydroxycarboxylic acids | S-protected thiolic acid |
| 69 | Polyserin | Combination of diol and dicarboxylic acid | Unprotected amino acid |
| 70 | Polyserin | Combination of diol and dicarboxylic acid | N-protected amino acid |
| 71 | Polyserin | Combination of diol and dicarboxylic acid | Unprotected thiolic acid |
| 72 | Poiyserin | Combination of diol and dicarboxylic acid | S-protected thiolic acid |
| 73 | Polyserin | Amino acid | Unprotected thiolic acid |
| 74 | Polyserin | Amino acid | O-protected thiolic acid |
| 75 | Polyserin | Combination of diamine and dicarboxylic acid | Unprotected thiolic acid |
| 76 | Polyserin | Combination of diamine and dicarboxylic acid | S-protected thiolic acid |

preferably

| | | | |
|---|---|---|---|
| 77 | Polyvinyl alcohol | Lactic acid | Unprotected amino acid |
| 78 | Polyvinyl alcohol | Lactic acid | N-protected amino acid |
| 79 | Polyvinyl alcohol | Lactic acid | β-alanine |
| 80 | Polyvinyl alcohol | Lactic acid | N-FMOC-β-alanine |
| 81 | Polyvinyl alcohol | Glycolic acid | Unprotected amino acid |
| 82 | Polyvinyl alcohol | Glycolic acid | N-protected amino acid |
| 83 | Polyvinyl alcohol | Glycolic acid | β-alanine |
| 84 | Polyvinyl alcohol | Glycolic acid | N-FMOC-β-alanine |
| 85 | Polyvinyl alcohol | Tartaric acid | Unprotected amino acid |
| 86 | Polyvinyl alcohol | Tartaric acid | N-protected amino acid |
| 87 | Polyvinyl alcohol | Tartaric acid | β-alanine |
| 88 | Polyvinyl alcohol | Tartaric acid | N-FMOC-β-alanine |
| 89 | Polyvinyl alcohol | Citric acid | Unprotected amino acid |
| 90 | Polyvinyl alcohol | Citric acid | N-protected amino acid |
| 91 | Polyvinyl alcohol | Citric acid | β-alanine |
| 92 | Polyvinyl alcohol | Citric acid | N-FMOC-β-alanine |
| 93 | Polyacrylic acid | Lactic acid | Unprotected amino acid |
| 94 | Polyacrylic acid | Lactic acid | N-protected amino acid |
| 95 | Polyacrylic acid | Lactic acid | Aminoethanol |
| 96 | Polyacrylic acid | Lactic acid | N-FMOC-colamine |
| 97 | Polyacrylic acid | Glycolic acid | Unprotected amino acid |
| 98 | Polyacrylic acid | Glycolic acid | N-protected amino acid |
| 99 | Polyacrylic acid | Glycolic acid | Aminoethanol |
| 100 | Polyacrylic acid | Glycolic acid | N-FMOC-colamine |
| 101 | Polyacrylic acid | Tartaric acid | Unprotected amino alcohol |
| 102 | Polyacrylic acid | Tartaric acid | N-protected amino alcohol |
| 103 | Polyacrylic acid | Tartaric acid | Aminoethanol |
| 104 | Polyacrylic acid | Tartaric acid | N-FMOC-colamine |
| 105 | Polyacrylic acid | Citric acid | Unprotected amino alcohol |
| 106 | Polyacrylic acid | Citric acid | N-protected amino alcohol |
| 107 | Polyacrvlic acid | Citric acid | Aminoethanol |
| 108 | Polyacrylic acid | Citric acid | N-FMOC-colamine |

more preferably

| | | | |
|---|---|---|---|
| 79 | Polyvinyl alcohol | Lactic acid | β-alanine |
| 80 | Polyvinyl alcohol | Lactic acid | N-FMOC-βalanine |
| 95 | Polyacrylic acid | Lactic acid | Aminoethanol |
| 96 | Polyacrylic acid | Lactic acid | N-FMOC-colamine |

According to a preferred embodiment of the present invention all of the particles of the invention described above are nanoparticles and accordingly have a length of <5 μm, preferably <3 μm, and more preferably <2 μm and/or a thickness and width of <200 nm, preferably <75 nm, and more preferably <30 nm.

By nanoparticle we mean a particle measuring less than 1 μm in at least two dimensions. In particular, nanoparticles have a volume of less than 1 m³. Nanoparticles are solid colloidal particles.

In a particularly preferred embodiment of the invention the particles of the invention are surface-modified in a specific manner. These particles are extremely well-suited for achieving the object of the invention since they are particularly suitable for effecting targeted transfer. Thus the invention also relates to particles for transportation of pharmaceutically active substances, to which linker molecules containing a reactive group (z′) selected from groups capable of forming a covalent bond with one of the groups (z) selected from the aforementioned “free groups” (z) selected from the group comprising OH, SH, COOH, and NH₂, and the vinyl or epoxide group, preferably an amino or thiol-reactive group, particularly an amino-reactive group, are covalently bonded through (z′)-(z) bonds with groups (z) present on the surface of the particle, said groups (z) being selected from the “free groups”. These “free groups” (z) on the surface of the particles of the invention are made available by the side-chain terminators (optionally following removal of the protective group).

By linker molecules we mean polymers, particularly unbranched or not more than triple-branched polymers which are capable of modifying the properties, particularly the surface properties, of the particle, but which in particular serve to effect sterically favorable bonding of other bio-active compounds to the particles or optionally sterically protect the particles from degradation.

By “reactive group” [(z′) and also any other (z″)] we mean particularly hitherto known groups which readily form a covalent bond with the aforementioned “free groups” (z), particularly amino, thiol, carboxy, or hydroxy groups, and also with epoxy or vinyl groups.

Preference is given to linker molecules which are bifunctional and have, in addition to the reactive group (z′) binding the particle of the invention, another reactive group (z″) at another end of the molecule, said other reactive group (z″) being selected from reactive groups capable of forming a covalent bond with one of the groups (z) selected from the group comprising the “free groups” (z) selected from the group comprising OH, SH, COOH, and NH₂, and the vinyl or epoxide group, preferably a thiol-reactive group, where z′≠z″.

According to another preferred embodiment of the invention, the thiol-reactive group, for example, binds to the particle of the invention and (in the case of bifunctional linkers), there is, for example, an amino-reactive group at another end of the linker molecule. The choice of the reactive groups of the linker molecules depends on the one hand on the free group(s), preferably free groups, on the particle of the invention to which the linker binds. On the other hand, it depends on a possible further modification or on the surface property imparted by the linker. In particular, a possible second reactive group on the linker molecule will be determined by the nature of a molecule possibly still to be formed or already formed (by synthesis) on the linker.

In another preferred embodiment, the linker molecules are a mixture of the bifunctional molecules described above and monofunctional molecules which have no other functional group (z″) of different reactivity at either end of the molecule, in addition to the group (z′) binding to the particle of the invention, preferably an amino-reactive or thiol-reactive group, particularly an amino-reactive group, where z′≠z″.

These particles are very advantageous, since bulky radicals such as antibodies can be readily taken on by the bifunctional radicals, whilst the monofunctional linkers sterically prevent any degradation. These particles are also referred to as acanthospheres.

In another preferred embodiment, distinctly more, preferably at least 100% more, monofunctional molecules than bifunctional molecules are covalently bonded to the surface of these particles (acanthospheres).

In a preferred embodiment, bio-active macromolecules or “seeker”molecules, selected from the group comprising peptides, proteins; preferably antibodies, antibody fragments, or antibody derivatives with target-binding properties such as“single-chain” antibodies; hormones, sugars, preferably glycosides; synthetic or natural receptor ligands; proteins or peptides having a free cysteine group, or thiosugars, are coupled or will be coupled or have already been coupled, prior to surface modification, to the bifunctional linker molecules via a bond to the reactive group (z″).

The term “seeker” molecules generally signifies, for the purposes of the present invention, compounds that are capable of being coupled to the particles of the invention and capable of binding with a high degree of affinity to the biological targets of the active substances, examples thereof being proteins, peptides, polysaccharides, oligosaccharides, lipoproteins, glycoproteins, or other biological molecules that are expressed either in healthy tissue (physiologically) or in or near diseased tissue (pathologically). Seeker molecules can be, for example, peptides, proteins, for example, antibodies, antibody fragments, or antibody derivatives having target-binding properties, such as “single-chain” antibodies; hormones, sugars, for example, glycosides; or synthetic or natural receptor ligands. Special preference is given to antibodies or derivatives or fragments thereof and glycosides.

According to another preferred embodiment, bio-active macromolecules or general “seeker” molecules, preferably antibodies, antibody fragments, or antibody derivatives having target-binding properties such as“single-chain” antibodies, particularly those containing a free cysteine group, are coupled, will be coupled or have already been coupled, prior to surface modification, to the bifunctional linker molecules via a bond to the reactive group (z″). This particularly applies to particles which have a coating containing distinctly more monofunctional molecules than bifunctional molecules. Irrespective of the order of the reactions there is thus obtained a completely covalently bonded macromolecule showing the following architecture: a central axis of polyvinyl alcohol (or some other high molecular weight polymer), from the OH groups (or corresponding functional groups) of which hydrophobic polycondensates of hydroxycarboxylic acids (or other condensable monomers) extend and terminate with non-hydroxycarboxylic acids (or other suitable terminators), which either end freely in hydrophilic groups or are coupled to polymeric, more-hydrophilic linkers, to some of which “seeker” molecules are attached.

In a preferred embodiment, bio-active micromolecules or seeker molecules, preferably sugars, particularly thiosugars, hormones, or proteins, particularly those containing a free cysteine group, are coupled, will be coupled, or have already been coupled, prior to surface modification, to the bifunctional linker molecules via a bond to the reactive group (z″). This applies, in particular, to particles having a coating consisting predominantly or completely of bifunctional molecules.

In another preferred embodiment, reactive groups (z″) still free on the particles of the invention after binding the bio-active micromolecules or “seeker” molecules are saturated with, preferably, cysteine.

In yet another preferred embodiment of the surface-modified particles, the linker molecules are monofunctional molecules which do not have at any end of the molecule any other group (z″) of different reactivity besides the reactive group (z′) binding to the particle of the invention, preferably an amino-reactive or thiol-reactive group, particularly an amino-reactive group, where z′≠z″.

Very preferably, the linker molecules are polyglycolides, preferably polyethylene glycol derivatives, particularly NHS-ester-PEG or NHS-ester/vinylsulfone-PEG.

Any subsequent purification (cleaning) or isolation is preferably effected by way of dialysis, preferably using selective exclusion membranes.

For all particles of the invention it is advantageous when the pharmaceutically active substance to be transported is a synthetic or natural active substance, a protein, peptide, lipid, sugar, or nucleic acid, or a low-molecular organic active substance or high-molecular organic active substance, for example, a hormone, a carcinostatic, an antibiotic, an antifungal agent, a parasiticide, a virustatic agent, or an antihelminthicum, a substance showing cardiovascular activity, or a substance acting on the central nervous system, particularly an analgesic, antidepressant, or antiepileptic.

Generally, a preferred embodiment of the particles of the invention is given when the particle of the invention is coupled directly or via a linker, preferably via bifunctional polyethylene glycol molecules, to a “seeker” molecule selected from the group comprising:

peptides, proteins; preferably antibodies, antibody fragments, or antibody derivatives showing target-binding properties such as “single-chain” antibodies; hormones, sugars, preferably glycosides; synthetic or natural receptor ligands; proteins or peptides containing a free cysteine group, or thiosugars.

In addition, processes for the production of particles of the invention are an important part of the invention. Thus the invention also relates to a process for the production of a particle of the invention in which an unbranched or at most triple-branched polymer backbone composed of monomers each having at least one bonding group (x) on each monomer is brought into contact with

monomers each having at least one bonding group (y) and at least one bonding group (x′), where (x′) is a group capable of forming a covalent bond both with (x) and with (y),

a mixture of monomers, of which at least one monomer exhibits at least two bonding groups (y) and at least one different monomer has at least two bonding groups (x′), where (x′) is a group which is capable of forming a covalent bond both with (x) and with (y), or

a mixture of monomers, of which at least one monomer exhibits at least two bonding groups (y), at least one different monomer has at least two bonding groups (x′) and at least one further different monomer has at least one bonding group (y) and at least one bonding group (x′), where (x′) is a group which is capable of forming a covalent bond both with (x) and with (y),

and at least one side-chain terminator, containing at least one bonding group (y′) and no bonding group (y) and at least one free group (z) optionally provided with a protective group, in which

the molar ratio of the monomers in the molecular side chains (b) to the side-chain terminators (c) is >>1,

group y≠group z and group x′≠group z,

the molar ratio of the monomers of the molecular backbone to the side-chain terminators (c) is approximately equimolar,

x, x′, y, and y′ are independently selected from the group comprising OH, SH, COOH, and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide linkage NH—C(O)—), SH/COOH (thioester bond —S—C(O)—), COOH/OH (ester bond —O—C(O)—), COOH/NH₂ (amide linkage —NH—C(O)—), and COOH/SH (thioester bond S—C(O)—),

z is selected from the group comprising CH₃, OH, SH, COOH, NH₂, and the vinyl or epoxide group, optionally protected by a protective group and

the side-chain monomers can be used as pure monomers or derivatives such as inner anhydrides or lactones provided they are still capable of forming chains with themselves and/or with other side-chain monomers,

under conditions which permit bonding between the monomers or monomer mixtures in the side chain and the polymer backbone and the side-chain terminators and permit polycondensation of the monomers or monomer mixtures in the side chain,

said contacting being carried out optionally in an anhydrous organic solvent, preferably pyridine, optionally in the presence of thionyl chloride and the particles are then optionally cleaned, preferably by dialysis against H₂O, and optionally isolated,

and the particles are then optionally dissolved, together with the hydrophobic or hydrophobed active substance to be transported, in an anhydrous organic solvent, and the solution is then incubated for a period of time, preferably overnight, and preferably at room temperature, after which the solution is saturated with water and the water-saturated solution is then dissolved in a larger volume of water, followed, optionally, by mechanical treatment and, optionally, cleaning of the particles, preferably by dialysis against H₂O, followed by isolation of the particles.

The invention also relates to a process for the production of a particle of the invention (containing linker molecules) in which a particle of the invention (not containing linker molecules) containing a group (z) selected from the “free groups” is brought into contact with a linker molecule containing a reactive group (z′) which is capable of forming a covalent bond with group (z), under conditions suitable for the formation of said covalent bond, after which the particles are optionally cleaned, preferably by dialysis against H₂O, and isolated. Suitable conditions are, for example, neutral to weak alkaline conditions.

The invention also relates to a process for the production of a particle of the invention (containing a linker molecule and a “seeker”molecule) in which, following execution of the above process, the particles thus produced (containing linker molecules) having a free reactive group (z″) on bifunctional linker molecules is brought into contact, under suitable conditions, with bio-active macromolecules or“seeker”molecules as defined above under conditions suitable for the formation of a bond between group (z″) and the bio-active macromolecules or“seeker” molecules, after which the particles are optionally cleaned, preferably by dialysis against H₂O, and isolated.

The particles of the invention are particularly suitable forms for increasing the desired effects of known active substances and for minimizing systemic side effects and by means of which controlled and/or spatially defined release of the effector molecules is achieved. For this reason they are suitable and intended for inclusion in a great variety of therapeutic agents. Thus the invention also relates to medicinal drugs containing particles of the invention and, optionally, suitable additives and/or auxiliaries.

Basically, the medicinal drugs of the invention can be administered as liquid pharmaceutical dosage forms in the form of aerosols, injection fluids, drops, or juices, or as semisolid pharmaceutical dosage forms in the form of granules, tablets, pellets, or capsules.

Suitable additives and/or auxiliaries are, for example, solvents or diluents, stabilizing agents, suspending agents, buffering agents, preserving agents, and also dyes, fillers, and/or binding agents. Selection of the adjuvants and of the amounts thereof to be used depends on whether the medicinal drug is to be administered, eg, by inhalation, orally, perorally, parenterally, intravascularly, intravenously, intraperitoneally, rectally, subcutaneously, or intramuscularly. Suitably preparations for oral administration are those in the form of tablets, dragees, capsules, granules, or suspensions such as drops, juices, and syrups, and for other administration purposes suspensions and readily reconstructable dry preparations are suitable.

The particles of the invention are also particularly suitable for use as diagnostic reagents, since they can place tags, for example, in precisely the desired cell. The invention thus also relates to a diagnostic reagent which contains the particle of the invention and, optionally, suitable additives and/or auxiliaries.

Furthermore,—as stated above—the particles of the invention are particularly suitable forms by means of which an increase in effectiveness and minimization of the side effects are achieved by the controlled and/or spatially defined release of the effector molecules so that the particles are generally useful for the production of therapeutic agents and they are by nature generally suitable for an unrestricted number of indications. Without the intention of restricting the use of the particles of the invention, mention may be made of their use for particular indications. The invention therefore also relates to the use of the particles of the invention for the production of a medicinal drug for the treatment of cancer, for the treatment of infectious diseases and parasitic diseases, for the treatment of diseases and symptoms caused by the central nervous system, for use in genetic therapy, or for genomic targeting. Other preferred applications include, for example, their use in targeting cytostatic drugs in tumour cells, the transfer of therapeutically useful substances through the blood-brain barrier, and the treatment of severe infections (specifically by eukaryotes).

Other possible applications are, for example, the transportation of vegetable alkaloids having a microbicidic action to trypanosomes and the transportation of antioxidants and anti-inflammatory compounds [vitamin E, colic acid, N-acetyl-L-cysteine, 2,6-bis(tert-butyl)4-mercaptophenol, ibuprofen, and gentisic acid] in the case of (degenerative) brain disorders, the transportation of substances to hepatocytes, primarily for the treatment of neoplasms, and causing an increase in the action of primaquine on plasmodium hypnozoites surviving in liver cells. The particles of the invention are also effective against Trypanosoma brucei brucei.

Examples of particularly suitable possible applications for the particles of the invention are:

transfer of otherwise non-brain-penetrating pharmacological agents (eg, cytostatic drugs, psychotropic agents, analgesics, M1 Alzheimer therapeutics) containing antibody-conjugated supports through the blood-brain barrier

targeted introduction of pharmacological agents (eg, virustatica, cytostatic drugs, plasmodicides) containing glycoside-conjugated supports into hepatocytes

Oral administration of otherwise only parenterally available pharmacological agents by targeting to intestinal epithelia

Increasing the action of antiparasitic therapeutic agents by targeting to parasite-specific surface molecules

Another embodiment of the process comprises the treatment of a human being or an animal requiring such treatment with or using a particle of the invention. This treatment is particularly suitable for the aforementioned indications and types of administration.

The invention is illustrated below with reference to examples, to which it is not restricted,

### FIGURES

FIG. 1 shows diagrammatically the general structure and the shape of particles of the invention, simple particles, simple stealth particles, target-seeking actinospheres, and target-seeking acanthospheres.

FIGS. 2a and 2b illustrate the synthesis of ktenates. The resultant molecule (a˜4000, b˜100) has a comb-like architecture, leading to a “bottle brush” shape. It is capable of forming, without an additional protective colloid, monomolecular particles having a mol. wt. of >30,000 kDa, between the side chains of which low-molecular substances can be trapped. The exposed amino groups of these particles can react with NHS-esters and in this way be linked with PEG-spikes.

FIG. 3 illustrates the overall structure of a finished monomolecular particle with spikes, a “bdellosome” (from the Greek bdella=leech) comprising lactic-β-alanine ktenate.

Length: ca 1800 nm

Spike length: ca 20 nm

Ø: unladen (not loaded) ca 4 nm

Loading efficiency: up to 80%

Stability: high

Surface coating: PEG (mol. wt.=3400), via NHS-ester bonded to terminal amino groups in the ktenate

Ligands: bonded via vinyl sulfone to distal ends of the PEG; eg, BSA, IgG (FIG.) and antibody fragments, transferrin, etc.

Load: eg, alkaloids, daunomycin

FIG. 4 shows an elektronic-microscopic photograph of BSA-conjugated ktenate particles (acanthospheres).

FIG. 5 shows the results of a model experiment on the monocellular parasite Trypanosoma brucei. Binding of particles manufactured by the process of the invention to target cells correlates with parasiticide action of the daunomycin contained therein.

### EXAMPLES

### General Remarks

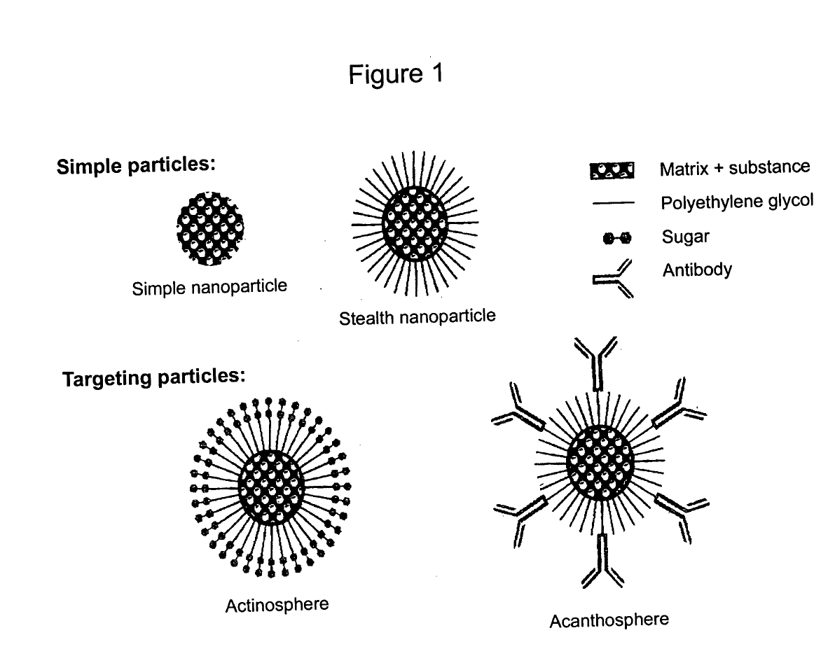

The following examples describe particles of the invention, particularly nanoparticles, which provide the possibility of embedding active substances in colloidal carrier particles. These might be linked, for example, to antibodies against, or natural ligands for, characteristic molecular structures of the target or to other “seeker molecules”. For example, the nanoparticles may at the same time be optionally protected against the immune system by an inert coating on their surface. Generally the particles of the invention described herein by way of example are colloidal, lipid-free particle systems. Some of the presently described particles of the invention are designated below by the general term actinosphere or acanthosphere (see FIG. 1). These terms are maintained irrespective of the actual geometry, since they refer to structural concepts and not to basic steric forms.

By reason of their shape, the particles are generally designated as bdellosomes.

### Example 1

### Production of the Basic Bdellosome Body

### a) General Description of the Body

The example is based on monomolecular particles of a polylactide derivative having a complex structure, referred to as a ktenate by reason of its comb-like structure. In aqueous environment ktenates form threadlike particles, so-called bdellosomes, of freely selectable dimensions achieved by variation of the synthesis parameters (diameter in the nanometer range, length up to several microns), the term bdellosome coming from the Greek “bdella”, which means “leech”. They are capable, on the one hand, of allowing stable embedding of low-molecular, preferably hydrophobic substances, therein, and, on the other hand, of being chemically modified on exposed functional groups such that “targeting” can be achieved. Realization of the particle systems is effected by synthesis of monomolecular particles based on polyvinyl alcohol as “backbone”, to the OH groups of which chains of polymeric lactic acid (or some other suitable hydroxycarboxylic acid) are added by condensation. Consequently the length of the polyvinyl backbone determines the size of the particle in one dimension (long axis) and is designated below as a.

Since the side chains consist of bifunctional monomers, each of which has a terminal OH group, to which the next acid molecule (either a monomer of the same kind or a chain-terminating molecule of a non-hydroxycarboxylic acid) can attach itself, and a terminal COOH group at the other end, which group can react with another hydroxyl group (either that of a monomer of the same kind or a chain-terminating OH group of the polyvinyl backbone), it is possible to provide, by the addition of small but mutually equimolar amounts of free alcohol and non-hydroxylated carboxylic acid, “terminators”, the concentration of which relatively to the concentration of the hydroxycarboxylic acid regulates the chain length. The terminator that is at the COOH end is the alcohol group in the polyvinyl backbone, and the terminator at the OH end is some other carboxylic acid. Of course, the molarity of the carboxylic acid to be attached at the OH end must correspond to the molarity of the OH groups in the polyvinyl alcohol in order to provide defined reaction conditions. Consequently, there results the following ratio b, which corresponds to the average chain length of the side chains and together with a gives the geometry of the resulting nanoparticle:

b=c(hydroxycarboxylic acid): c(OH groups)=c(non-hydroxycarboxylic acid)

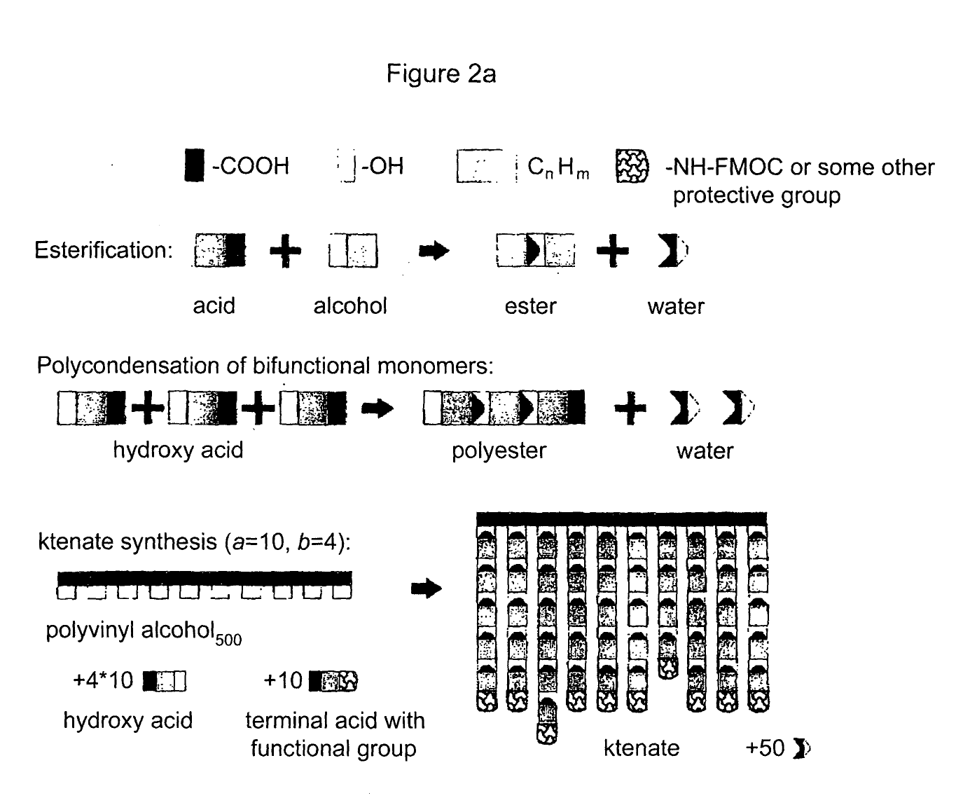

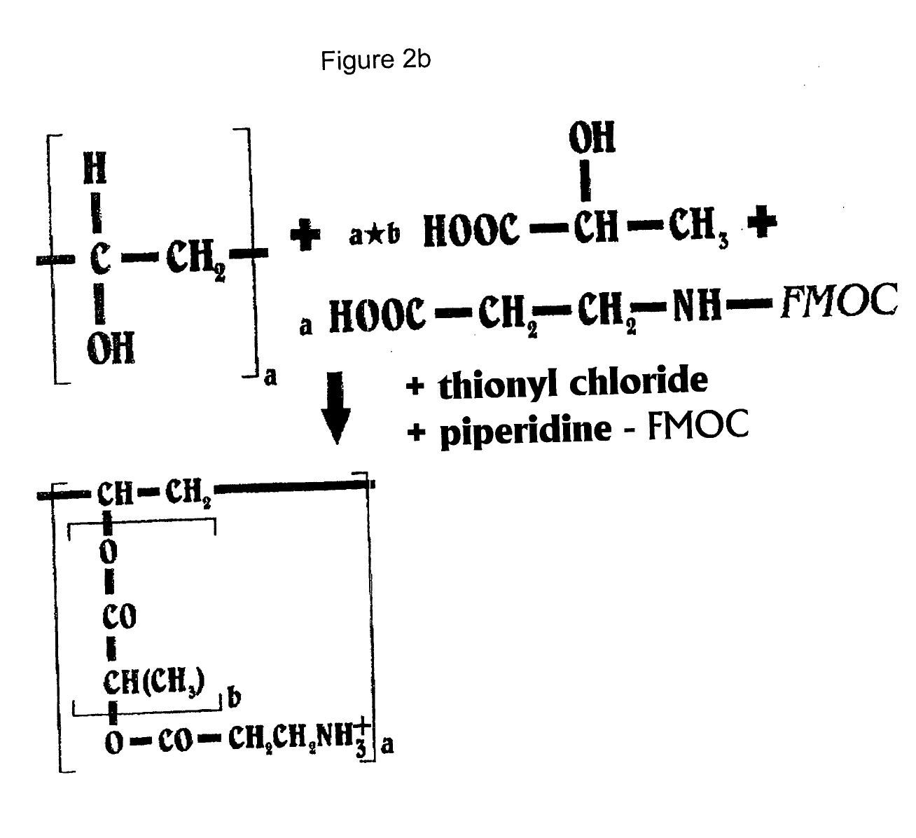

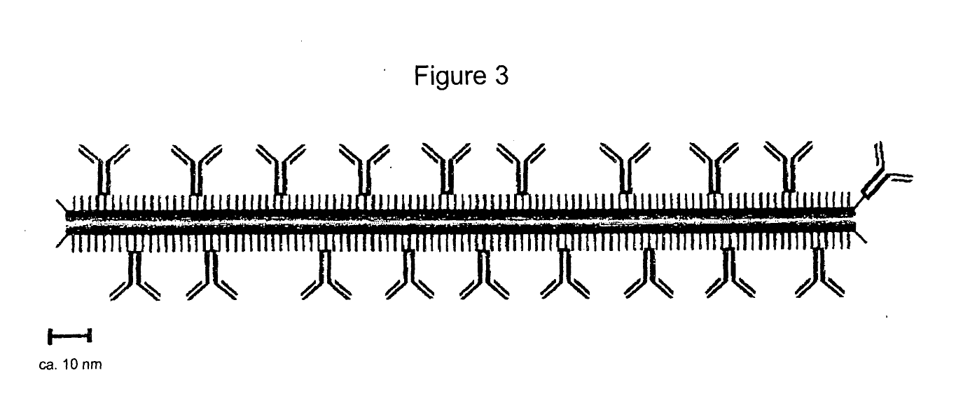

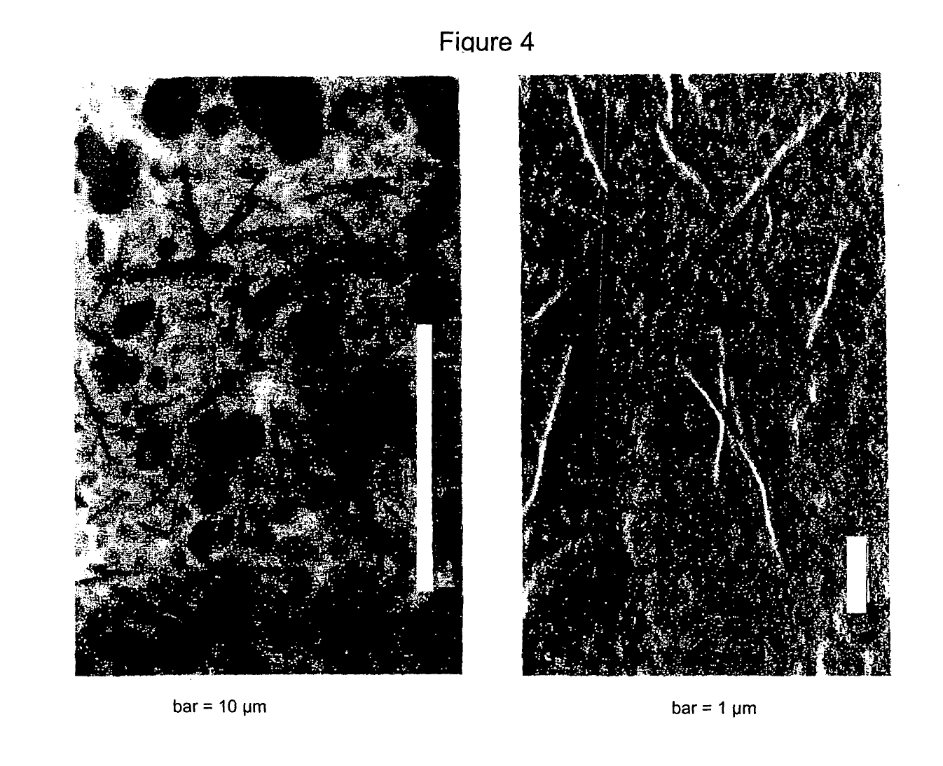

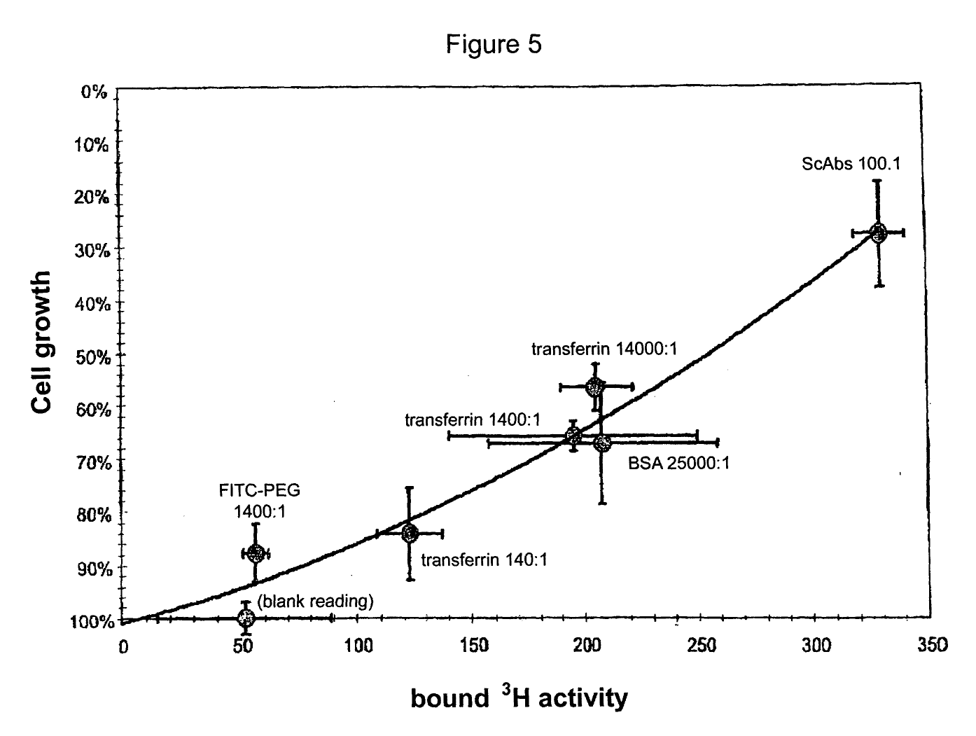

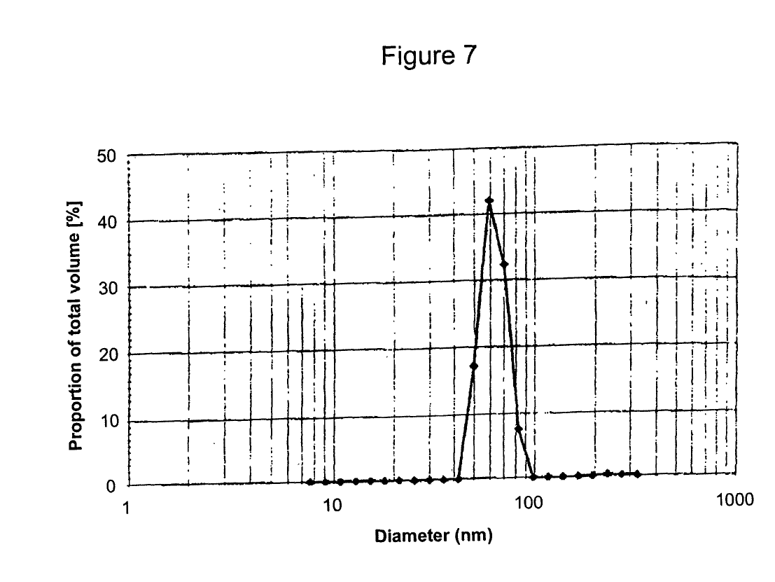

Synthesis is carried out by causing the reaction of a reaction mixture of polyvinyl alcohol, hydroxycarboxylic acid and non-hydroxycarboxylic acid in anhydrous environment with thionyl chloride, which converts the acid groups to the corresponding chlorides, which then react with hydroxyl groups with elimination of water to form polycondensates (cf. FIG. 2).

There results a molecule similar to a comb, as represented diagrammatically, and having a side chains which hang down from the polyvinyl backbone and have an average length of b monomer units each terminated by a non-hydroxylated terminator. The selection of suitable monomers leads to an arrangement which is energetically unfavorable in an aqueous environment and causes the molecular structure to “roll up” to form a three-dimensional structure, in which the side chains project all round the backbone (“bottle brush” effect). The side chains stretch out the backbone substantially so that the dimensions of the nanoparticle vary within the following range:

Length=a*length of the polyvinyl monomer

Width and height=*length of the side-chain monomer

If the terminator of the side chains is an amino acid protected by, say, FMOC, the protective group can be eliminated at the end of the synthesis reaction (in the case of FMOC by treatment with catalytic amounts of piperidine), by which means side chains having a terminal amino group spatially exposed in an aqueous environment are obtained, which not only stabilize the structure and prevent flocculation in the aqueous medium on account of their hydrophilicity but also can serve as starting points for surface reactions (cf Example 2).

### b) Synthesis Procedure

Molecules were produced by causing suitable amounts of polyvinyl alcohol 200,000 (Mowiol™), lactic acid, and N-FMOC-β-alanine to react with thionyl chloride using pyridine as solvent followed by cleavage of the FMOC group with piperidine, which molecules can be designated as polyvinyl(te/o-alanylpolylactide)ate[a=4000, b=100]. This family of compounds will be referred to below by the general designation acid-terminator-ktenate(a,b) (Greek kteis=comb).

The finished lactalanine ktenate is washed with dichloromethane and then, without further cleaning, dissolved in a suitable volume of benzyl alcohol together with the substance of interest. The solution is incubated overnight at room temperature, then saturated with water and finally taken up in a larger volume of water. Without any further mechanical treatment (supersonics), the droplets of organic phase disintegrate within of few hours and form a homogeneous suspension of substance-loaded ktenate particles.

Due to the elongated shape of the ktenate particles, statements on the dimensions of the particle population are naturally difficult to make, but the QELS process could show that >95% w/w of a typical preparation (unfiltered) was present in the form of particles in the submicron range.

### Example 2

### Synthesis of the Actinospheres and Acanthospheres

### a) General Notes

Both in actinospheres and in acanthospheres, the other components comprise a bifunctional linker, particularly a polyethylene glycol molecule, which carries an amino-reactive group at one end and a different group of different reactivity at the other end. This bifunctional polyethylene glycol prevents, on the one hand, access of the immune system to the particles due to steric blockage of the surface with an inert molecule (as similarly tested in connection with liposomal artifacts by the procedure known as “stealth technology”) whilst also causing a greater degree of stabilization, and, on the other hand, affords the possibility of attaching to the particles further molecules capable of imparting the desired target orientation, said attachment being effected at a spatially favorable and flexible site on the particles. These molecules can be micromolecules, eg, sugar, in which case exclusive use of bifunctional polyethylene glycol is possible (actinospheres), or they can be macromolecules such as antibodies (acanthospheres), in which case it is advisable, for steric reasons,to cause only a small portion of the functional groups on the particle surface to react with bifunctional polyethylene glycol and to saturate the rest with monofunctional polyethylene glycol, which then merely serves to effect physical and immunological stabilization of the particles.

### b) Synthesis Procedure following on Example 1

In order to suppress immune reactions, the finished bdellosomes of Example 1 were covalently linked via the functional surface groups to “spikes” of polyethylene glycol (mol. wt. ca 3400 dalton), to the distal ends of which there can in turn be attached antibodies or other molecules useful for targeting purposes.

Conjugation of the particles with “spikes” of NHS-ester-PEG is carried out by simple mixing and incubation at room temperature, preferably in a weakly alkaline medium, followed by cleaning by dialysis against a 500-fold volume of water (at a mol. wt. cut-off of 12-14 kDa, both non-bonded PEG-spikes and non-conjugated ktenate particles can escape, but not ktenate-PEG-conjugates).

When use is made of bifunctional NHS-ester-PEG-vinylsulfone spikes, there follows a secondary conjugation of the ktenate-PEG complex with the “seeker” proteins, followed by saturation of any coupling groups which may still be free with cysteine (approx. 1 mg of cysteine per mg of PEG equivalent to a 20-30-fold molar excess) and a second cleaning step, again by dialysis, this time using a dialysis membrane having an appropriately higher mol. wt. cut-off.

Finally, it is generally recommended to saturate any unsaturated free coupling groups with cysteine or some other SH reagent. Thus it is recommendable, following the reaction of the bdellosome-PEG-conjugates with the “seeker” molecules, to saturate any coupling groups still free at the distal ends of the PEG-spikes with a high molar excess of a suitable reactant, for example cysteine, in the case of vinyl sulfone. This prevents these coupling groups from reacting with endogenic molecules (eg, serum proteins) in the organism, which would falsify the target orientation.

When use is made of the slow-reacting, relatively water-stable vinyl sulphone group as the distal reaction group of the PEG-spikes, it is possible to prepare these thiophilic (ktenate) particles for all applications by a standardized method and to couple them with suitable “seeker molecules” after they have been cleaned, which molecules can be of arbitrary chemical nature and are only required to possess a sulfydryl group so that actinospheres and acanthospheres can be produced. In the case of acanthospheres, simple in-process verification of the final linking step can be achieved by adding fluorescein-tagged or self-fluorescent proteins (GFP) or by western blotting of random samples, and, in the case of actinospheres, by carrying out confirmatory tests in thin-layer chromatography.

### Example 3

### Properties of Bdellosomes Produced According to Example 1

The stability and packing efficiency of bdellosomes are exceptionally high compared with other colloidal packing systems. The loading efficiency was examined with reference to the cytostatic drug daunomycin, which has already been used on a clinical basis. Using the model substance daunomycin, packing rates of over 80% of the material used were achieved. Using a small portion of ³H-tagged daunomycin, a yield of up to 99% of the total amount used was achieved.

No signs of decomposition or flocculation of the particles were discernable following storage over a period of many months at room temperature. In addition, the simplicity of the synthesis of the bdellosomes resulting from the monomolecular structure of the particle is particularly noteworthy. The size and shape of the particles could be made visible in the scanning electron microscope after weak gold deposition and fulfilled expectations (cf FIG. 4); when use was made of BSA, which possesses several free thiol groups, slight crosslinking of the particles occurred, as expected. In the case of bdellosomes covalently linked to “spikes” of polyethylene glycol (mol. wt. 3400) via the functional surface groups, to the distal ends of which spikes there may in turn be attached antibodies or other molecules useful for targeting, there was found to be a reduction in the undesirable uptake of the particles by the reticulo-endothelial system in the rat model.

### Example 4

### Efficiency of Bdellosomes Produced as Described in Example 2

Control of the parasitic protozoon Trypanosoma brucei brucei by means of acanthospheres loaded with daunomycin

### a) Choice of the Model Organism

Trypanosoma brucei brucei is a protozoic (Ord. Kinetoplastida) parasite, which is itself not human pathogenic but is of direct economical significance both on account of the Nagana epidemic caused thereby in African livestock and due to the fact that it can be used as a laboratory model for the control of the closely related human pathogenic forms Trypanosoma cruzi (Chagas' disease, >20 million infected persons in South America), Trypanosoma brucei gambiense and Trypanosoma brucei rhodesiense (sleeping sickness, very prevalent in the population of central Africa) and also some smaller trypanosome species (T. equinum, T. equiperdum, T. evansi) and the closely related leishmanial diseases (Leishmania donovani, germ causing kalaazar or visceral leishmaniasis; L. tropica, which causes of oriental sore; L. brasiliensis, mucocutaneous leishmaniasis).

The treatment of parasitic protozoa is generally difficult and is based predominantly on suramin, pentamidine and organic arsenic and antimony compounds such as melarsoprol(QUERY) and stibophen, which show poor compatibility in the concentrations required for therapy. Moreover the trypanosomes migrate in the late stage of contamination to the central nervous system, where they are substantially not affected by chemotherapy due, to the blood-brain barrier of the host. Thus trypanosomes are doubly suitable as a model, on the one hand for direct targeting and on the other hand for transportation through the blood-brain barrier. The following is a description of direct targeting.

Trypanosomes evade the immune system of the host by varying their cell surface proteins by recombination, to which only some receptors (for example for transferrin and albumin) are an exception, but these are positioned in valleys of the cell surface such that antibodies binding thereto cannot initiate any immune reaction to cause destruction of the parasite cell. However, it is theoretically possibly to couple bdellosomes loaded with cytotoxins to ligands for these receptors and to cause adequate endocytization thereof.

### b) Synthesis of Acanthospheres based on Bdellosomes

For both binding and cytotoxizity studies use was made of bdellosome suspensions (0.5 g of lactalanine ktenate 4000, 100 per liter) loaded with 1% (m/m) of daunomycin (equivalent to a concentration of 10⁻⁵ mol of daunomycin per liter of suspension) exhibiting a tritium label of ca 1000 cpm/μg. Free daunomycin causes inhibition of the growth of trypanosomes upwards of a concentration of 10⁻⁷ mol in the medium.

The synthesis of the particles was carried out as described in Example 1. As described in Example 1, the bdellosomes were linked via their superficial amino groups to monofunctional PEG or coupled via said amino groups and bifunctional (NHS-ester-vinylsulfone)-PEG having an average molecular weight of 3400 dalton to cysteine radicals of various proteins: human transferrin in various concentrations, bovine serum albumin, and single-chain-antibodies counteracting the transferrin receptor. Finally, free coupling groups were saturated with cysteine.

### c) Binding Study

In each case, ˜3*10⁵ of parasites showing good growth were incubated for 20 min at room temperature with a 1:1 mixture of one of the aforementioned bdellosome suspensions and growth medium, after which the supernatant liquor was removed and the cells were washed in isotonic solution of common salt and finally taken up in fresh salt solution. The tritium activities in the supernatant liquor, washing buffer, and cellular fraction were determined by scintillation counting.

### d) Cytotoxicity Study

In each case, 10 μl of the aforementioned bdellosome suspensions were added to a suspension of ca 10⁵ of parasites showing good growth in fresh nutrient medium (such that a final concentration of 10⁻⁷ mol was achieved) and incubated overnight under growth conditions and then counted after a period of 24 hours, and the number of parasite cells was compared with that of the controls treated only with an isotonic solution of common salt.

### e) Conclusion

The results of the binding and cytotoxicity studies show a correlation between cytotoxicity and binding of >97%. The strongest-binding fraction (bdellosomes linked with antibodies) reduced the cell density of the parasites to a quarter of that found in the controls treated only with isotonic solution of common salt, ie, a pronounced cytotoxic effect was observed. In the absence of “seeker” proteins, however, but using the same daunomycin concentration, neither binding nor cytotoxicity could be observed.

In a further aspect the invention relates to solid particles for transportation of pharmaceutically active substances, to processes for the preparation thereof, to medicinal drugs containing said particles, and to the use of said particles in various specific indications.

A main goal of pharmaceutical research is to increase the desired effects of known active substances and to minimize systemic side effects, which is of great significance for substances exhibiting high intrinsic and thus unavoidable toxicity (eg, cytostatic drugs). This can be achieved both by reducing the total dosage required for the desired therapeutic effect and by causing accumulation of the effectors at the desired site of action, both of which possibilities may be realised by controlled, spatially specified release of effector molecules in the widest sense (proteins, peptides, nucleic acid, or low-molecular substances) in the desired target tissue. By this is meant, in particular, the specific transportation of therapeutically or diagnostically useful substances to defined biological targets (“drug delivery”, “drug targeting”), which is an important aim of present-day pharmaceutical research.

Present-day antibody technology allows for the production of highly affinic bonding partners for almost any biological structure; moreover numerous natural ligands have been characterized and cloned for cellular receptors so that it is no longer problematic to produce molecules showing high and specific affinity toward the desired targets. In many cases low-molecular ligands (eg, glycosides) are also known which can be imitated by chemical means and thus used for targeting. However these “seeker molecules” whether micromolecular or macromolecular, generally exhibit no pharmaceutically useful function, while the effectors themselves are not target-oriented. Thus it must be a main goal to overcome this discrepancy and to combine the therapeutic potential of the available effectors with the target orientation of the seeker molecules.

The most efficient attempt at achieving this goal known hither consists in the use of carrier structures of a colloidal (ie submicron) order of magnitude, to the surfaces of which appropriate target-seeking molecules can be attached. In this way there are achieved both optimal ratios of target-seeking molecules to effector molecules and maximum flexibility. Whereas only a few (<10) effector molecules can be bonded to a single antibody molecule or other ligand molecule by direct covalent attachment (which means that for a molecular weight of an antibody of approx. 150 kDa more than 90% of the mass of the conjugate is accounted for by the antibody moiety), and conjugates with low-molecular seekers usually exhibit a molar ratio of 1:1, the use of colloidal systems makes an effector to target-seeker ratio of 10³-10⁴ possible. Moreover no chemical modification of the effector is required, which is advantageous in every respect.

One efficient way of achieving this is to embed the relevant substances in colloidal carrier particles, which are linked with antibodies against, or with natural ligands for, characteristic molecular structures of the target and are at the same time protected against the immune system by an inert coating on their surface.

A much used method of colloidal packing of pharmacological agents consists in enclosing the effectors in lipid membrane-coated vesicles (liposomal artifacts or liposomes). The use of appropriate membrane components makes it possible, on the one hand, to bind the desired target-seeking molecules to the liposomal membranes, and, on the other hand, to coat the supports with anti-immunogenic material (eg, polyethylene glycol) and by this means to afford protection from non-specific removal from the bloodstream by the reticulo-endothelial system. The advantages of this system are offset by the following serious drawbacks:

The thermal and aging stability of the vesicle consisting of a single double lipid layer is limited, as is its impermeability. Theoretically, the permeability of the membranes to hydrophilic materials can be reduced, but the required modified (eg, fluorinated) lipids are not biologically acceptable. Moreover one single “direct hit” of the complementary system will suffice to cause a vesicle to run out completely.

The poor stability of the membrane vesicle in turn restricts the possibilities of varying the surface structure and thus limits the number of potential applications.

Only hydrophilic substances can be transported in the interior aqueous phase of the vesicle in adequate concentration.

Loading the liposomes is carried out (apart from a few special cases) by simply enclosing a portion of the aqueous phase and is accordingly inefficient. Typically <0.5% of the effector substance is enclosed in the vesicle. During this process the substance is exposed to considerable thermal and chemical stresses (the operating temperature must be above the critical phase transition temperature of the lipid mixture for a considerable time, and the reactive groups required for the covalent modification survive this only when the pH is very low).

Half of the membrane reactive groups required for the linkage with proteinaceous seeker molecules are, following vesicle formation, on the inside of the vesicle and are not available for linking purposes, but, when the liposomes have been assimilated, said membrane reactive groups are liberated into the organism and can lead to unforeseeable reactions.

Chemical coupling of proteins to liposomal membranes (eg, via SPDP linked directly, or through a polyethylene glycol arm, to lipids) leads to the formation of highly immunogenic structures, which have been used successfully for vaccination purposes but which must be regarded as being useless in the field of “drug targeting”, since they cause an immune reaction against the particles.

An alternative consists in the use of solid colloidal particles (“nanoparticles”). Nanoparticles are basically well known. Particles of the micrometric and submicrometric order of magnitude and comprising hydrophobic polymers can theoretically be produced by finely dispersing the polymer taken up in a non-polar solvent. Removal of the solvent causes the polymer to precipitate in the form of particles having a diameter smaller than that of a droplet; loading with hydrophobic substances (into which category most of the pharmacological agents fall) can be effected simply by adding the substance to the non-polar solvent. Following removal of the solvent, the active substance is approximately 100% associated with the polymer and remains, when the particles are present in an aqueous phase, attached to the particle matrix not by covalent bonds but by van der Waals' forces and steric entrapment, this attachment being stable for a relatively long period. An essential feature in this case is that subsequent coagulation of the hydrophobic particles (whose large surface of contact with the hydrophilic medium is energetically unfavorable) is prevented, for example by synthesizing the particles in the absence of an amphiphilic substance, which mediates between the hydrophobic particle matrix and the hydrophilic medium.

Conventional nanoparticles and the preparation thereof and possibilities of surface variation are disclosed in WO 96/20698, the system here being particularly optimized for intravascular use, especially for treatment of restenosis. The Patent Application describes polymolecular nanoparticles of natural or synthetic polycondensates and contains a predominantly general description of a property-modifying surface coating of natural or synthetic macromolecules.

The the following documents may also be mentioned by way of example.

DE 19,810,965 A1 describes polymolecular nanoparticles of a polyelectrolyte complex of polycations and polyanions which is treated with a crosslinking agent.

DE 198 39 515 A1 describes colloidal polymer/active substance associates with a property-optimizing branched polyol-ester for use on mucous tissues.

U.S. Pat. No. 6,117,454 describes, in particular, polymolecular nanoparticles which are made suitable for penetration of the blood-brain barrier by a coating of fatty acid derivatives.

U.S. Pat. No. 5,840,674 describes complexes of active substance and microparticle which are permanently covalently attached via a “linker”, particularly for use against microorganisms.

U.S. Pat. No. 5,641,515 describes polymolecular nanoparticles of polycyanoacrylate containing insulin, which effect controlled release of the coordinated insulin.

Hitherto known nanoparticles and methods of synthesizing the same often exhibit problems regarding stability and producibility and also as regards the extent to which loading is possible, and can therefore be used to a limited extent only. In particular, the problem of achieving precise administration of the active substance at the desired site of action and of attaining stability of the nanoparticles from the time of administration to their arrival at the site of action, ie the problem of “drug targeting” is inadequately solved. A central problem relates to the surface modification of nanoparticles. As mentioned above, conventional solid nanoparticle systems constitute, unlike liposomal artifacts, an energetically unfavorable system by reason of their extremely high specific interphase between the hydrophobic particle matrix and the hydrophilic medium, which is unstable in the absence of stabilizing agents. Conglomeration of the particles minimizes the energetically unfavorable surface of contact, but this leads to precipitation of the hydrophobic particulate substance.

For this reason, conventional nanoparticles require a coating of, for example, amphiphilic molecules, which lower the interfacial energy and in this way stabilize the particles. This coating covers the particle matrix completely and allows no access of modifying agents thereto. On the other hand, modification effected by just such amphiphilic molecules leads, however, to a drastic change in the physical properties and thus to destabilization of the coating. For this reason, is it scarcely possibly to modify conventional nanoparticles such that binding of ligands and thus the use thereof in the field of drug targeting is possible. The solutions hitherto offered in the prior art treat surface modification either very generally or are restricted to a particular problem to such an extent that more general use is not possible or the solution given constitutes in itself a disadvantage.

It is thus an object of the invention to provide nanoparticles which do not exhibit the above drawbacks and, in particular, are capable of allowing specific release at the site of action.

This object is achieved by means of solid particles for the transportation of hydrophobic or hydrophobed pharmaceutically active substances, said particles being synthesized by a process involving the following steps:

(a) the preparation of a solution in an organic solvent or a mixture of organic solvents containing at least one hydrophobic or hydrophobed pharmaceutically active substance, water-insoluble organic polymeric material, amphiphilic organic polymeric material, and, optionally, supplementary materials,

(b) treatment of the solution with supersonics,

(c) dialysis of the solution against H₂O

(d) separation of the resulting particles from the resulting aqueous solution.

The particles of the invention are particularly suitable forms for achieving an increase in the action and minimization of the side effects by the controlled and/or spatially specific release of the effector molecules. Generally the particles involved in the invention constitute preferably solid, colloidal and/or lipid-free particle systems.

By hydrophobed active substances we mean active substances which were originally more hydrophilic but have been rendered hydrophobic by chemical modification. As an example there may be mentioned hydrophobic resorption esters of hydrophilic active substances.

When synthesizing said nanoparticles of the invention, it is particularly preferred that the organic solvent(s) are soluble in water in a ratio of solvent to water of from 1:10 to 1:50, preferably from 1:20 to 1:40, and more preferably from 1:20 to 1:30, and/or are preferably selected from the group comprising:

dichloromethane or benzyl alcohol, preferably benzyl alcohol.

However, the selection of a suitable solvent is by no means limited to dichloromethane and benzyl alcohol.

Furthermore, it is preferred that during production of the particles of the invention the organic polymeric material insoluble in water is dissolved together with the hydrophobic pharmaceutically active substance(s), and the amphiphilic organic polymeric material obtained is first of all separated and optionally dissolved together with the supplementary material(s), and the solutions are only then mixed for production of the solution according to step (a).

In another preferred embodiment of the process for production of the particles of the invention, the separately dissolved amphiphilic organic polymeric material and/or any co-dissolved supplementary material are present, prior to mixing with the non-polar polymeric material, in a concentration between 10 and 45% (w/v), preferably between 20 and 40% (w/v), and more preferably 35% (w/v).

It is also preferred that the organic polymeric material insoluble in water is present prior to step (b) in a concentration between 3 and 0.1% (w/v), preferably between 2 and 0.5% (w/v), and more preferably 1.7% (w/v).

It is further preferred that the particles of the invention are produced by a process in which the solution in step (b) is treated with supersonics for a period ranging from 1 h to 15 h, preferably from 4 h or 5 h to 10 h, particularly from 5 h to 6 h. The ultrasonic treatment is preferably effected at maximum power output.

It is also preferred that the concentration ratio in % (w/v) of the organic polymeric material insoluble in water of step (a) and the amphiphilic polymeric material of step (b) is, on conclusion of step (b), between 1:2 and 1:32, preferably between 1:4 and 1:28, and more preferably between 1:8 and 1:20, very preferably between 1:12 and 1:16, and most preferably 1:14.

For the production of the above particles of the invention it is particularly preferred that the water-insoluble organic polymeric material of step (a) is selected from the group comprising

polyesters, preferably polylactic acid (polylactide), polyglycolide or polylactide/polyglycolide copolymers (PLGA), particularly pure polylactide, pure polypropylene glycol, or polylactide/polyglycolide copolymer (for example, in a ratio of 3:1).

However, for the synthesis of the particles of the invention use may also be made of other biocompatible, degradable synthetic polymers such as appropriate polyanhydrides, polyamino acids, polycyanoacrylates, polyacrylamides, or polyurethanes.

When synthesizing the said particles of the invention it is preferred that the amphiphilic organic polymeric material of step (a) is selected from the group comprising

polyvinyl alcohol or derivatives of polyvinyl alcohol, preferably pure polyvinyl alcohol, esters of polyvinyl alcohol and a hydrophobic carboxylic acid (preferably a fatty acid) or co-esters in which each molecule of polyvinyl alcohol is esterified with at least one hydrophobic carboxylic acid (preferably a fatty acid) and a second, different carboxylic acid.

When synthesizing the above particles of the invention is it particularly preferred that the supplementary material(s) are selected from the group comprising

alkali metal salts or alkaline earth metal salts of organic acids, particularly magnesium salts, preferably magnesium acetate.

It is also preferred that the process steps (a) to (c) take place at physiological temperatures, preferably between 35° and 40° C., particularly at 37° C.

Another embodiment fulfilling the object of the invention relates to solid particles for transportation of hydrophobic or hydrophobed pharmaceutically active substances, which have a core of organic polymeric material insoluble in water and an outer layer of amphiphilic polymeric material which is non-covalently linked to the molecules of the nucleus, said amphiphilic polymeric material being selected from the group comprising

polyvinyl alcohol or esters of polyvinyl alcohol.

It is particularly preferred that the particles contain non-covalently linked hydrophobic or hydrophobed pharmaceutically active material.

It is also preferred that these particles of the invention contain amphiphilic polymeric material selected from the group comprising

pure polyvinyl alcohol, esters of polyvinyl alcohol and a hydrophobic carboxylic acid (preferably a fatty acid) or co-esters, in which each molecule of polyvinyl alcohol is esterified with at least one hydrophobic carboxylic acid (preferably a fatty acid) and a second, different carboxylic acid.

For all of the aforementioned particles of the invention, a particularly preferred embodiment is one in which the amphiphilic organic polymeric material used is selected from the group comprising co-esters of polyvinyl alcohol, in which each molecule of polyvinyl alcohol is esterified with at least one hydrophobic carboxylic acid (preferably a fatty acid) and a second, different carboxylic acid,

the hydrophobic carboxylic acid preferably being selected from the group comprising fatty acids having a length of between 10 and 24 carbon atoms, preferably unsubstituted or not substituted with COOH, OH, SH, or NH₂, particularly not substituted with COOH or OH, preferably selected from C₁₀-C₁₆ fatty acids, particularly lauric acid,

the second, different carboxylic acid being selected from the group comprising

carboxylic acids, preferably not substituted by COOH or OH and preferably substituted by SH or NH₂, preferably NH₂, particularly amino acids, preferably alanine,

preferably selected from the group comprising co-esters in which polyvinyl alcohol is esterified with at least one fatty acid and at least one amino acid, particularly poly(vinyllaurate-co-β-alanate), preferably poly(vinyllaurat(25%)-co-β-alanate(7%)).

For all particles of the invention described above, a preferred embodiment of the present invention is one in which the particles are nanoparticles and/or exhibit in at least two dimensions a length of 10 to 500 nm, preferably <150 nm, and more preferably from 50 to 100 nm.

By nanoparticles we mean, in a narrower sense of the present invention, particles which exhibit a length below 1 μm in each dimension, or, in a broader sense, particles which exhibit a length below 1 μm in at least two dimensions. The narrower definition particularly encompasses all particles having a volume below 1 μm³, preferably 0.01 μm³, and more preferably 0.0001 μm³. Nanoparticles are solid colloidal particles.

Surface-modified particles are, in particular, a central feature of the present invention and achieve the object of the invention in an excellent manner. Thus the invention also relates to particles for the transportation of pharmaceutically active substances, to which linker molecules containing an amino-reactive and/or thiol-reactive, preferably an amino-reactive, group are covalently bonded via free NH₂ or SH groups, preferably NH₂ groups, which were previously present on the surface of the particle (ie prior to modification of the particle with the linkers).

By linker molecules we mean polymers, particularly unbranched polymers, which are capable of modifying the properties, particularly the surface properties, of the particle, but which serve, in particular, to effect sterically advantageous bonding of other bio-active compounds to the particles or possibly also to protect the particles sterically from degradation.

By the term “reactive groups” we refer, in particular, to hitherto known groups which readily bind to amino, thiol, or hydroxy groups, and also epoxy and vinyl groups.

Particular preference is given to an embodiment in which the linker molecules are bifunctional and exhibit not only an amino-reactive or thiol-reactive, preferably amino-reactive, group, but also another functional group of different reactivity, preferably a thiol-reactive group, at another end of the molecule.

Another preferred embodiment is one in which the linker molecules constitute a mixture of bifunctional molecules—as described above—and monofunctional molecules carrying only either an amino-reactive or a thiol-reactive group, but preferably an amino-reactive group.

Another preferred embodiment of the invention relates to particles for the transportation of pharmaceutically active substances in which a mixture of two types of linker molecules is present on the surface of the particle, which molecules are covalently bonded at one end of the linker molecules via a reactive group to the surface of the particle, the first (bifunctional) type of linker molecule carrying at at least one other end of the molecule another reactive group, whilst the second (monofunctional) type of linker molecule carries no further reactive groups at any other end of the molecule.

Both of the aforementioned groups of particles are very favorable, since they allow bulky radicals such as antibodies to be added unhindered to the bifunctional radicals, whilst the monofunctional linkers sterically prevent degradation. These particles are also referred to as acanthospheres.

It is again particularly preferred when there are covalently bonded to the surface of the two aforementioned types of particle (acanthospheres) distinctly more, preferably at least 100% more, monofunctional molecules than bifunctional molecules.

In particular, preference is given to an embodiment in which the linker molecules are polyglycolides, preferably polyethylene glycol derivatives, and more preferably NHS-ester-PEG or NHS-ester/vinylsulfone-PEG.

Another preferred embodiment is one in which the particles of the invention provided with linkers are nanoparticles.

Furthermore, preference is given to said particles which are particles of the invention as described above without reference to linkers.

A preferred embodiment is one in which there are coupled, there will be coupled, or there have been coupled, prior to surface modification, to the bifunctional linker molecules bio-active macromolecules or “seeker”molecules, selected from the group comprising peptides, proteins; preferably antibodies, antibody fragments, or antibody derivatives showing target-binding properties such as“single-chain” antibodies; hormones, sugars, preferably glycosides; synthetic or natural receptor ligands; proteins or peptides having a free cysteine group, or thiosugars.

For the purposes of the present invention, the term “seeker” molecule generally refers to compounds that are capable of being coupled to the particles of the invention and which are capable of binding with high affinity to the biological targets of the active substances, which might be proteins, peptides, polysaccharides, oligosaccharides, lipoproteins, glycoproteins, or other biological molecules expressed either in healthy tissue (physiologically) or in or near diseased tissue (pathologically).

“seeker”molecules may be, for example, peptides, proteins, for example, antibodies, antibody fragments, or antibody derivatives showing target-binding properties such as“single-chain” antibodies; hormones, sugars, for example, glycosides; or synthetic or natural receptor ligands. Special preference is given to antibodies or derivatives or fragments thereof, and glycosides.

Another preferred embodiment is one in which there are coupled, there will be coupled, or there have been coupled, prior to surface modification, to the bifunctional linker molecules bio-active macromolecules or, generally speaking, “seeker” molecules, preferably antibodies, antibody fragments, or antibody derivatives showing target-binding properties such as “single-chain” antibodies, particularly those having a free cysteine group. This particularly applies to particles which have a coating containing distinctly more monofunctional molecules than bifunctional molecules.

Another preferred embodiment is one in which there are coupled, there will be coupled, or there have been coupled, prior to surface modification, to the bifunctional linker molecules bio-active micromolecules or “seeker” molecules, preferably sugars, particularly thiosugars, hormones or proteins, particularly those having a free cysteine group. This applies, in particular, to particles having a coating consisting predominantly or completely of bifunctional molecules.

Another preferred embodiment is one in which any reactive groups still free on the particles of the invention after bonding of the bio-active micromolecules or “seeker” molecules are saturated, preferably with cysteine.

Any subsequent cleaning or isolation is preferably effected by way of dialysis, preferably using selective exclusion membranes.

For all of the particles of the invention it is preferred that the pharmaceutically active substance to be transported is a synthetic or natural active substance, a protein, peptide, lipid, sugar, or nucleic acid or a low-molecular organic active substance or high-molecular organic active substance, for example, a hormone, a carcinostatic, an antibiotic agent, antifungal agent, parasiticide, virustatic agent, or antihelminthicum, a substance showing cardiovascular activity; a substance acting on the central nervous system, particularly an analgesic, antidepressant, or antiepileptic.

A generally preferred embodiment of all of the particles of the invention is one in which the particle is coupled, directly or via a linker, preferably via bifunctional polyethylene glycol molecules, to a “seeker” molecule selected from the group comprising:

peptides, proteins; preferably antibodies, antibody fragments, or antibody derivatives showing target-binding properties such as “single-chain” antibodies; hormones, sugars, preferably glycosides; synthetic or natural receptor ligands; proteins or peptides having a free cysteine group, or thiosugars.

The use of bifunctional polyethylene glycol molecules for the surface modification of particles for transportation of pharmaceutically active substances is a particularly important feature of the present invention. Thus this invention also relates to the use of pure bifunctional polyethylene glycol molecules, preferably NHS-ester/vinylsulfone-PEG; or mixtures of bifunctional and monofunctional polyethylene glycol molecules, preferably NHS-ester-PEG with NHS-ester/vinylsulfone-PEG; for the production of surface-substituted solid particles for transportation of pharmaceutically active substances.

A particularly preferred embodiment is one in which, via the bifunctional polyethylene glycol molecules, “seeker” molecules selected from the group comprising

peptides, proteins; preferably antibodies, antibody fragments, or antibody derivatives showing target-binding properties such as single-chain antibodies; hormones, sugars, preferably glycosides; synthetic or natural receptor ligands; proteins or peptides having a free cysteine group, or thiosugars

are bonded to the particles, preferably when use is made of pure bifunctional polyethylene glycol molecules, glycosides, particularly thiosugars; or when use is made of mixtures of bifunctional and monofunctional polyethylene glycol molecules, antibodies, antibody fragments, or antibody derivatives showing target-binding properties such as single-chain antibodies, preferably antibodies having a free cysteine group.

In addition, processes for the production of particles of the invention are an important part the invention. Thus the invention also relates to a process for the production of a particle of the invention, particularly the second of the aforementioned types of particle containing PLGA (polylactide/polyglycolide), which comprises the following steps:

(a) the preparation of a solution in an organic solvent or in a mixture of organic solvents containing at least one hydrophobic pharmaceutically active substance, water-insoluble organic polymeric material and amphiphilic organic polymeric material,

(b) treatment of the solution with supersonics,

(c) dialysis of the solution against H₂O

(d) separation of the resulting particles from the resulting aqueous solution.

The particles of the invention are particularly suitable forms for increasing the desired effects of known active substances and for minimizing systemic side effects and by means of which controlled and/or spatially defined release of the effector molecules is achieved. For this reason they are suitable and intended for inclusion in a wide variety of therapeutic agents. Thus the invention also relates to medicinal drugs containing particles of the invention and, optionally, suitable additives and/or auxiliaries.

Basically, the medicinal drugs of the invention can be administered as liquid pharmaceutical dosage forms in the form of aerosols, injection fluids, drops, or juices, or as semisolid pharmaceutical dosage forms in the form of granules, tablets, pellets, or capsules.

Suitable additives and/or auxiliaries are, for example, solvents or diluents, stabilizing agents, suspending agents, buffering agents, preserving agents, and also dyes, fillers, and/or binding agents. Selection of the adjuvants and of the amounts thereof to be used depends on whether the medicinal drug is to be administered, eg, by inhalation, orally, perorally, parenterally, intravascularly, intravenously, intraperitoneally, rectally, subcutaneously, or intramuscularly. Suitable preparations for oral administration are those in the form of tablets, dragees, capsules, granules, or suspensions such as drops, juices, and syrups, and for other administration purposes suspensions and readily reconstructable dry preparations are suitable.

As stated above, the particles of the invention are particularly suitable forms, by means of which an increase in action and minimization of side effects can be achieved by the controlled and/or spatially specific release of the effector molecules so that these particles are useful in a general manner for the production of therapeutic agents and they are naturally generally suitable for an unrestricted number of indications. Without the intention of restricting the use of the particles of the invention, mention may be made of their use for particular indications. The invention therefore also relates to the use of the particles of the invention for the production of a medicinal drug for the treatment of cancer, for the treatment of infectious diseases and parasitic diseases, for the treatment of diseases and symptoms caused by the central nervous system, for use in genetic therapy, or for genomic targeting. Other preferred applications include, for example, their use in targeting cytostatic drugs in tumour cells, the transfer of therapeutically useful substances through the blood-brain barrier, and the treatment of severe infections (specifically by eukaryotes).

Other possible uses comprise, eg, the transportation of vegetable alkaloids having a microbicidic action into trypanosomes and the transportation of antioxidants and anti-inflammatory compounds [vitamin E, colic acid, N-acetyl-L-cysteine, 2,6-bis(tert-butyl)-4-mercaptophenol, ibuprofen and gentisic acid] in the case of (degenerative) brain disorders, the transportation of substances into hepatocytes, primarily for the treatment of neoplasms, and also increasing the action of primaquine on the plasmodium hypnozoites surviving in the liver cells.

Examples of particularly suitable possible applications for the particles of the invention are:

the transfer of otherwise non-brain-penetrating pharmacological agents (eg, cytostatic drugs, psychotropic agents, analgesics, M′ Alzheimer therapeutics) containing antibody-conjugated supports through the blood-brain barrier

the targeted introduction of pharmacological agents (eg, virustatica, cytostatic drugs, plasmodicides) containing glycoside-conjugated supports into hepatocytes

oral administration of otherwise only parenterally available pharmacological agents by targeting onto intestinal epithelia

increasing the action of antiparasitic therapeutic agents by targeting to parasite-specific surface molecules.

Another embodiment of the process comprises the treatment of a human being or an animal requiring such treatment with or involving a particle of the invention. This treatment is particularly suitable for the aforementioned indications and types of administration.

The invention is illustrated below with reference to examples to which it is not restricted.

### EXAMPLES AND DRAWINGS

### FIGURES

FIG. 1 shows diagrammatically the general structure and the shape of particles of the invention, simple particles, simple stealth particles, target-seeking actinospheres, and target-seeking acanthospheres.

FIG. 7 shows the uniformity of the size distribution of particles synthesized according to the invention as described in Example 1.

### EXAMPLES

### General Remarks

The following examples describe particles of the invention, particularly nanoparticles, which provide the possibility of embedding active substances in colloidal carrier particles. These might be linked, for example, with antibodies against, or natural ligands for, characteristic molecular structures of the target or with other “seeker molecules”. For example, the nanoparticles may at the same time be optionally protected against the immune system by an inert coating on their surface. Generally the particles of the invention described herein by way of example are colloidal, lipid-free particle systems. Some the presently described particles of the invention are designated below by the general term actinosphere or acanthosphere (see FIG. 1). These terms are maintained irrespective of the actual geometry, since they refer to structural concepts and not to basic steric forms.

### Example 1

### Nanoparticles in General

These are particles of an order of magnitude ranging from 10 nm to 500 nm and comprising a solid core of organic water-insoluble polymeric material, which is coated with a layer of an amphiphilic organic polymer, which, by reason of its properties, is non-covalently but securely bonded to the water-insoluble particle matrix and both stabilizes the particles toward the aqueous environment and protects them from cohering and from flocculation which could result therefrom. Furthermore, the amphiphilic organic polymer is provided with reactive functional groups, which make it possible to couple other components thereto by chemical means.

The material used for the production of the nanoparticles of this example was polylactic acid (polylactide, PLA). Existing literature on the synthesis of particles in the order of magnitude of 1-10 μm (used for other purposes) confirms the suitability of this substance, which over and above most other polymers has the advantage of being biodegradable. It is also possible to provide PLA with more-hydrophilic terminal groups, which line up at the boundary layer to the aqueous medium during production and either themselves act as low-molecular target seekers or can be used as a point of attachment for proteinaceous target seekers.

Synthesis of nanoparticles was established with reference to the substance RG752 (sold by Boehringer Ingelheim), a block copolymer of 75% of polylactide and 25% of polyglycolide having an average mol. wt. of 12 kDA, and has been successfully used on the pure polylactide R202H (Boehringer) and polypropylene glycol 26000 (Sigma; liquid at room temperature). The respective polymer is dissolved in benzyl alcohol—possibly in the presence of the substance to be transported therein—(to give a final concentration of 5%), and this solution is mixed with twice its volume of a highly osmotic protective colloid (35% of polyvinyl alcohol and 35% of magnesium acetate). Many hours of ultrasonic treatment cause the dissolved polymer to be extremely finely dispersed. Dialysis of the resulting milky product against a large excess of water causes the removal of magnesium acetate and benzyl alcohol (solubility in

water 1:25) and also of excess polyvinyl alcohol so that an aqueous suspension of poly(vinyl alcohol)-coated nanoparticles remains.

Evaluation of the manufacturing process was carried out on the principle of “quasi-elastic light scatter” (QELS) in Zetasizer equipment (Zetasizer 3000 HS, sold by Malvern). It was found that after simple blending of dissolved polymer and protective colloid (agitator, vortex), liquid particles in the micron range are still present, and these particles change to nanoparticles only when subjected to ultrasonic treatment. In conformity with the model according to which the polyvinyl alcohol stabilizes the boundary surfaces, the polyvinyl alcohol concentration is the decisive factor governing the diameter of the resulting nanoparticle: polyvinyl alcohol in a concentration of 10% led to the formation of particles in the range of 300-400 nm, and in a concentration of 35% to particles of 50-100 nm. (The critical constant for colloidal systems is approx. 150 nm; particles below this size can theoretically—ie when linked to suitable seeker molecules—traverse the blood-brain barrier.) In all experiments, the temperature was kept substantially within the physiological range, that is to say, between 25° and 45° C.

The duration of the ultrasonic treatment has no noticeable influence on the particle size, but has a considerable influence on the yield of usable particles. Following treatment over a period of 15 min about 90% of the counted particles, which, however, together form only ca 1% of the entire mass of polylactide, are in the desired size range, whilst the rest consists of microparticles. Following a period of 1 h of sonication the nanoparticle fraction comprises ca 10% of the composition, and following a period of 5-6 h, between 70% and 99% of the entire polylactide has been converted to nanoparticles. The rest is present in two substantially discrete microparticle fractions, a smaller fraction showing diameters of from one half to one whole micron, and a larger fraction showing diameters of several microns, which could reflect a two-phase course of the dispersion prozess. Following a period of ca 15-18 h, degradation (noticeable by discoloration) and agglomeration began until finally, following a period of 24 h, the entire reaction mixture had degenerated to a wax-like composition.

In some measurements a secondary peak occurred in the range of 250-300 nm; however, dilution tests could show that this was caused merely by aggregates of the relatively hydrophobic particles. Moreover, with all of the tested substances higher particle densities give an apparent diameter of the individual particles that is 20-30% greater than the real diameter, since apparently interactions between the particles inhibit the movement thereof, which forms the basis of QUELS measurements. As was to be expected, both manifestations of this tendency to aggregate of the more hydrophobic substances R202H and polypropylene glycol in both high and low dilution were considerably more pronounced than in the case of the conjugate samples of nanoparticles of the glycolide-containing copolymer RG752.

The results of a typical measurement (1 volume of 5% RG752 in benzyl alcohol sonicated together with 2 volumes of 35% magnesium acetate/35% polyvinyl alcohol for a period of 6 hours at maximum power output and at 42° C., then dialyzed for a period of 48 hours against 600 volumes of water [exclusion size of the membrane 25-30 kDa, renew the water used for dialysis following a period of 24 hours]; all samples diluted with water 1:36 prior to measurement), are given below:

Unfiltered:

| | | |
|---|---|---|
| Peak | Area under curve | Average diameter |

According to signal intensity:

| | | |
|---|---|---|
| #1 | 59.4% | 48.2 nm |
| #2 | 22.5% | 240.5 nm |
| #3 | 5.8% | 749.6 nm |
| #4 | 12.2% | 2045 nm |

According to proportion of total volume:

| | | |
|---|---|---|
| #1 | 88.4% | 49.9 nm |
| #2 | 1.1% | 748.2 nm |
| #3 | 9.6% | 2542 nm |

According to number of particles:

| | | |
|---|---|---|
| #1 | 100.0% | 48.4 nm |

Following filtration through an 800 nm filter (see FIG. 2)

The size distribution observed after filtration is comparable to that of a good preparation of liposomal artifacts, but the advantage lies in the dissimilarly higher packing efficiency.

It has been possible to modify the surface properties of the particles by linking the polyvinyl alcohol used as protective colloid to various organic acids. It was found to be essential to hydrophobe the polyvinyl alcohol, whose water solubility is at the upper limit for a coating substance, since the exclusive introduction of reactive groups increased the hydrophilicity of polyvinyl alcohol to such an extent that it simply passed into solution and no particle formation could take place. This could be achieved by partial esterification with fatty acids via the acid chlorides. The resulting polyvinyl laurate (20%) showed particle-binding properties superior to those of the original polyvinyl alcohol. Using comparable amounts of protective colloid essentially shorter sonication times were required with polyvinyl laurate to give higher yields of somewhat smaller particles.

In the next step, polyvinyl alcohol was caused to react simultaneously with different amounts of lauryl chloride and p-alanyl chloride to produce poly(vinyl laurate(25%)-co-β-alanate(7%)). This substance was comparable to polyvinyl laurate as regards particle-forming properties but provided large amounts of covalently bonded primary amino groups. By reason of the loose surface texture, which allows for compensation of the loads of NH₄⁺ groups by interposed acetate, no surface potential could be observed, but the existence of the amino groups bound to supermolecular structures could be definitely proven using chemical agents.

### Example 2

### Synthesis of Actinospheres and Acanthospheres

### a) General Notes

Both in actinospheres and in acanthospheres, the further components comprise a bifunctional polyethylene glycol molecule carrying at one end an amino-reactive group and at the other end a different group of different reactivity. This bifunctional polyethylene glycol prevents, on the one hand, access of the immune system to the particles due to steric blockage of the surface with an inert molecule (as similarly tested in connection with liposomal artifacts by the procedure known as “stealth technology”) whilst also causing a greater degree of stabilization, and, on the other hand, affords the possibility of attaching to the particles further molecules capable of imparting the desired target orientation, said attachment being effected at a spatially favorable and flexible site on the particles. These molecules can be micromolecules, eg, sugar, in which case exclusive use of bifunctional polyethylene glycol is possible (actinospheres), or they can be macromolecules such as antibodies (acanthospheres), in which case it is advisable, for steric reasons, to cause only a small portion of the functional groups on the particle surface to react with bifunctional polyethylene glycol and to saturate the rest with monofunctional polyethylene glycol, which then merely serves to effect physical and immunological stabilization of the particles.

Furthermore, if coupling groups should still be free at the distal ends of the PEG-spikes following the reaction of the nanoparticle-PEG-conjugates with the “seeker” molecules, it is recommendable to saturate any such groups with a high molar excess of a suitable reactant, for example, cysteine in the case of vinyl sulfone. This prevents these coupling groups from reacting with endogenic molecules (eg, serum proteins) in the organism, which would falsify the target orientation.

### b) Synthesis Procedure Following on Example 1

Following the synthesis of the nanoparticles as described in Example 1, they were surface-modified with polyethylene glycol. The reaction of the particles coated with poly(vinyl laurate(25%)-co-β-alanate(7%)) with NHS-ester/fluoroescein-polyethylene glycol in a neutral to weakly alkaline environment, followed by isolation of the non-bonded polyethylene glycol by dialysis of the suspension of particles against water produced “proto-actinospheres” whose properties could be examined with reference to their fluorescence and the physical properties of the polyethylene glycol molecules. It could be shown that a covalently bonded layer of fluorescein-tagged polyethylene glycol covered the surface of the nanoparticle in high molecular density (“self-quenching” of fluorescence) and to a depth of 10-15 nm (hydrated), as illustrated in the diagrammatic drawing relating to “actinospheres” (FIG. 1). The particle-forming properties of the poly(vinyl laurate-co-β-alanate) were distinctly superior to those of unmodified polyvinyl alcohol.

The reaction of the particle coated with poly(vinyl laurate(25%)-co-β-alanate(7%)) with NHS-ester/vinylsulfone-PEG in a weakly alkaline environment followed by clean-up by dialysis against water accordingly produces particles which carry thiol-reactive groups at the distal, substantially movable end of the polyethylene glycol “spikes” covalently bonded to the particle surface.

When use is made of the slow-reacting, relatively water-stable vinyl sulphone group as the distal reaction group of the PEG-spikes, it is possible to prepare these thiophilic particles for all applications by a standardized method and to combine them with suitable “seeker molecules” once they have been cleaned, which molecules can be of arbitrary chemical nature and need only possess a sulfhydryl group so that actinospheres and acanthospheres can be produced. In the case of acanthospheres, simple in-process verification of the final linking step can be achieved by adding fluorescein-tagged or self-fluorescent proteins (GFP) or by western blotting of random samples, and in the case of actinospheres by carrying out confirmatory tests in thin-layer chromatography.

### Example 3

### Efficiencies and Properties of Nanoparticles Produced as Described in Example 1 or Example 2

The load in the nanoparticles was tested by means of the relatively hydrophobic fluorescent dye 4-Di-10ASP, as used for staining cell membranes and belonging to the dialkylaminostyrenes (sold by Molecular Probes Inc.), and fulfilled the expectations based on the measured size distributions: approximately 90% of the fluorescent dye used was duly packed into nanoparticles. (In aqueous solution, uncharacterized physical effects interfered with the fluorescence inversely with the degree of dilution, so that the 4-Di-10ASP content could only be fluorimetrically determined following dissolution of the particles in an excess of a methanol/chloroform mixture.)

The structural stability of the particle was found to be remarkably high: without the addition of conserving substances or the like it was found that no significant change in the size distribution or 4-Di-10ASP load had occurred after eight weeks' storage at room temperature without the addition of further stabilizing substances.

With the nanoparticles covalently linked to “spikes” of polyethylene glycol (mol. wt. 3400) via the functional surface groups, at the distal ends of which “spikes” in turn antibodies or other molecules useful for targeting can be attached, there was found to be a reduction in the undesirable uptake of the particles by the reticulo-endothelial system on the rat model.

### Example 4

### Efficiency of Acanthospheres

Particles for transportation of pharmaceutically active substances having a volume of <1 μm³ and laden with tritium-tagged daunomycin were coupled, via their superficial amino groups, either to monofunctional polyethylene glycol (PEG) or to cysteine radicals of various “seeker” proteins, via bifunctional (NHS-ester/vinylsulfone)-PEG having an average molecular weight of 3400 dalton:

human transferrin in various concentrations,

bovine serum albumin, and

single-chain-antibodies counteracting the transferrin receptor.

Finally, free coupling groups were saturated with cysteine. A fraction of the particles not containing “seeker” molecules was also prepared.

The acanthospheres were incubated with parasites of the parasitic protozoon type Trypanosoma brucei brucei, and binding and cytotoxicity were examined.

The results of the binding and cytotoxicity studies show a correlation between cytotoxicity and binding. And the acanthospheres provided with “search” proteins reduced the cell density of the parasites distinctly compared with controls, ie, a pronounced cytotoxic effect was observed. In the absence of “seeker” proteins, however, but using the same daunomycin concentration, neither binding nor cytotoxicity could be observed.

Preferred embodiments of this aspect of the present invention are included in the following list of particular embodiments.

1. A solid particle for transportation of hydrophobic or hydrophobed pharmaceutically active substances, synthesized by a process comprising the following steps:

(a) preparation of a solution in an organic solvent or a mixture of organic solvents containing at least one hydrophobic or hydrophobed pharmaceutically active substance, water-insoluble organic polymeric material and amphiphilic organic polymeric material, and optionally supplementary substances,

(b) treatment of the solution with supersonics,

(c) dialysis of the solution against H₂O,

(d) separation of the resulting particles from the resulting aqueous solution.

2. A particle as defined in claim 1, characterized in that

the organic solvent(s) dissolve in water in a ratio of solvent to water of from 1:10 to 1:50, preferably from 1:20 to 1:40, and more preferably from 1:20 to 1:30, and/or are preferably selected from the group comprising:

dichloromethane or benzyl alcohol, preferably benzyl alcohol, and/or

the water-insoluble organic polymeric material is present, prior to step (b), in a concentration between 3 and 0.1% (w/v), preferably between 2 and 0.5% (w/v), and more preferably 1.7% (w/v), and/or

the solution is treated with supersonics in step (b) for a period ranging from 1 h to 15 h, preferably from 4 h or 5 h to 10 h, and more preferably from 5 h to 6 h, and/or

the concentration ratio in % (w/v) between the water-insoluble organic polymeric material of step (a) and the amphiphilic polymeric material of step (b) is, on conclusion of step (b), between 1:2 and 1:32, preferably between 1:4 and 1:28, more preferably between 1:8 and 1:20, very preferably between 1:12 and 1:16, and most preferably 1:14, and/or

the process steps (a) to (c) are carried out at physiological temperatures, preferably between 35° and 40° C., and more preferably 37° C., and/or

the water-insoluble organic polymeric material is dissolved together with the hydrophobic pharmaceutically active substance(s) and the amphiphilic organic polymeric material is first of all separated therefrom and dissolved, optionally with the supplementary material(s), and the solutions for the preparation of the solution as described under step (a) are only then mixed, it being preferred that,

the separately dissolved amphiphilic organic polymeric material and/or an optionally co-dissolved supplementary substance are present, prior to preparation of the mixture with the non-polar polymeric material, in a concentration between 10 and 45% (w/v), preferably between 20 and 40% (w/v), and more preferably 35% (w/v).

3. A particle as defined in claim 1 or claim 2, characterized in that the water-insoluble organic polymeric material of step (a) is selected from the group comprising

polyesters, preferably polylactic acid (polylactide), polyglycolide or polylactide/polyglycolide copolymers (PLGA), particularly pure polylactide, pure polypropylene glycol, or polylactide/polyglycolide copolymers (preferably in a ratio of 3:1). and/or

the amphiphilic organic polymeric material of step (a) is selected from the group comprising

polyvinyl alcohol or derivatives of polyvinyl alcohol, preferably pure polyvinyl alcohol, esters of polyvinyl alcohol and a hydrophobic carboxylic acid (preferably a fatty acid) or co-esters in which each molecule of polyvinyl alcohol is esterified with at least one hydrophobic carboxylic acid (preferably a fatty acid) and a second, different carboxylic acid, and/or

the supplementary substance(s) is/are selected from the group comprising

alkali metal salts or alkaline earth metal salts of organic acids, particularly magnesium salts, preferably magnesium acetate.

4. A solid particle for transportation of hydrophobic or hydrophobed pharmaceutically active substances, containing a core of organic water-insoluble polymeric material and an outer layer of amphiphilic polymeric material non-covalently bonded to the molecules of the core, characterized in that the amphiphilic polymeric material is selected from the group comprising

polyvinyl alcohol or esters of polyvinyl alcohol.

5. A particle as defined in claim 4, characterized in that the particles contain non-covalently bonded hydrophobic or hydrophobed pharmaceutically active substance.

6. A particle as defined in claim 4 or claim 5, characterized in that the amphiphilic polymeric material is selected from the group comprising

pure polyvinyl alcohol, esters of polyvinyl alcohol and a hydrophobic carboxylic acid (preferably a fatty acid) or co-esters, in which each molecule of polyvinyl alcohol is esterified with at least one hydrophobic carboxylic acid (preferably a fatty acid) and a second, different carboxylic acid.

7. A particle as defined in any one of claims 1 to 6, characterized in that the amphiphilic polymeric material is selected from the group comprising co-esters of polyvinyl alcohol in which each molecule of polyvinyl alcohol is esterified with at least one hydrophobic carboxylic acid (preferably a fatty acid) and a second, different carboxylic acid,

the hydrophobic carboxylic acid being preferably selected from the group comprising fatty acids having a length between 10 and 24 carbon atoms, which are preferably unsubstituted or are not substituted by COOH, OH, SH, or NH₂, particularly not by COOH or OH, and are preferably selected from C₁₀-C₁₆ fatty acids, particularly lauric acid, and/or

said second, different carboxylic acid being selected from the group comprising carboxylic acids which are preferably not substituted by COOH or OH but are preferably substituted by SH or NH₂, particularly NH₂, particularly amino acids, preferably alanine,

and is preferably selected from the group comprising co-esters in which polyvinyl alcohol is esterified with at least one fatty acid and at least one amino acid, particularly poly(vinyl laurate-co-β-alanate), and preferably poly(vinyl laurate(25%)-co-β-alanate(7%)).

8. A particle as defined in any one of claims 1 to 7, characterized in that the particles are nanoparticles and/or exhibit a length of from 10 to 500 nm, preferably <150 nm, and more preferably from 50 to 100 nm in at least two dimensions.

9. A particle for transportation of pharmaceutically active substances, characterized in that linker molecules containing an amino-reactive and/or thiol-reactive, preferably an amino-reactive, group are covalently bonded to the particles via NH₂ or SH groups, preferably NH₂ groups previously present as free groups on the surface of the particle.

10. A particle as defined in claim 9, characterized in that the linker molecules are bifunctional and contain, in addition to an amino-reactive or thiol-reactive, preferably amino-reactive, group, also another functional group, at another end of the molecule, which other group shows different reactivity and is preferably a thiol-reactive group.

11. A particle as defined in claim 9 or claim 10, characterized in that the linker molecules constitute a mixture of bifunctional molecules as defined in claim 10 and monofunctional molecules carrying only either an amino-reactive or a thiol-reactive, preferably an amino-reactive, group.

12. A particle for transportation of pharmaceutically active substances, characterized in that there is present on the surface of the particle a mixture of two types of linker molecule covalently bonded, at one end of the linker molecules, to the surface of the particle via a reactive group, the first (bifunctional) type of linker molecule carrying at at least one other end of the molecule another reactive group, whilst the second (monofunctional) type of linker molecule carries no further reactive groups at any other end of the molecule.

13. A particle as defined in claim 11 or claim 12, characterized in that there are covalently bonded to the surface of the particle distinctly more, preferably at least 100% more, monofunctional molecules than bifunctional linker molecules.

14. A particle as defined in any one of claims 9 to 13, characterized in that the linker molecules are polyglycolides, preferably polyethylene glycol derivatives, and more preferably NHS-ester-PEG or NHS-ester/vinylsulfone-PEG.

15. A particle as defined in any one of claims 9 to 14, characterized in that the particle is a nanoparticle.

16. A particle as defined in any one of claims 9 to 15, characterized in that the particle is as defined in any one of claims 1 to 8.

17. A particle as defined in any one of claims 9 to 14, characterized in that bio-active macromolecules or “seeker” molecules selected from the group comprising peptides, proteins; preferably antibodies, antibody fragments, or antibody derivatives showing target-binding properties such as “single-chain” antibodies; hormones, sugars, preferably glycosides; synthetic or natural receptor ligands; proteins or peptides having a free cysteine group, or thiosugars, are coupled, will be coupled, or have been coupled, prior to surface modification, to said bifunctional linker molecules.

18. A particle as defined in any one of claims 11 to 13, characterized in that bio-active macromolecules or “seeker” molecules, preferably antibodies, antibody fragments or antibody derivatives showing target-binding properties such as“single-chain” antibodies, particularly those containing a free cysteine group, are coupled, will be coupled, or have been coupled, prior to surface modification, to said bifunctional linker molecules.

19. A particle as defined in claim 10, characterized in that bio-active micromolecules or “seeker” molecules, preferably sugars, particularly thiosugars; peptides or hormones, particularly those containing a free cysteine group, are coupled, will be coupled, or have been coupled, prior to surface modification, to said bifunctional molecules in the outer coating.

20. A particle as defined in any one of claims 17 to 19, characterized in that any reactive groups still free after bonding of the bio-active micromolecules or “seeker” molecules are saturated, preferably with cysteine.

21. A particle as defined in any one of claims 1 to 20, characterized in that the active substance to be transported is a synthetic or natural active substance, a protein, peptide, lipid, sugar, or nucleic acid, or a low-molecular organic active substance or a high-molecular organic active substance, for example, a hormone, a carcinostatic, an antibiotic agent, an antifungal agent, a parasiticide, a virustatic agent, or an antihelminthicum, a substance showing cardiovascular activity, or a substance acting on the central nervous system, particularly an analgesic, antidepressant or antiepileptic.

22. A particle as defined in any one of claims 1 to 8, characterized in that it is directly coupled or is coupled via a linker, preferably via bifunctional polyethylene glycol molecules therein, to a “seeker” molecule selected from the group comprising:

peptides, proteins; preferably antibodies, antibody fragments, or antibody derivatives showing target-binding properties such as “single-chain” antibodies; hormones, sugars, preferably glycosides; synthetic or natural receptor ligands; proteins or peptides having a free cysteine group, or thiosugars.

23. A method of using pure bifunctional polyethylene glycol molecules; preferably NHS-ester/vinylsulfone-PEG; or mixtures of bifunctional and monofunctional polyethylene glycol molecules; preferably NHS-ester-PEG with NHS-ester/vinylsulfone-PEG; for the synthesis of surface-modified solid particles for transportation of pharmaceutically active substances.

24. A method as defined in claim 23, characterized in that, via said bifunctional polyethylene glycol molecules, “seeker” molecules selected from the group comprising

peptides, proteins; preferably antibodies, antibody fragments, or antibody derivatives showing target-binding properties such as “single-chain” antibodies; hormones, sugars, preferably glycosides; synthetic or natural receptor ligands; proteins or peptides containing a free cysteine group, or thiosugars

are bonded to the particles, preferably using pure bifunctional polyethylene glycol molecules such as glycosides, particularly thiosugars; or using mixtures of bifunctional and monofunctional polyethylene glycol molecules such as antibodies, antibody fragments, or antibody derivatives showing target-binding properties such as “single-chain” antibodies, preferably those containing a free cysteine group.

25. A process for the production of a particle as defined in any one of claims 4 to 6, characterized by the following steps:

(a) the preparation of a solution in an organic solvent or in a mixture of organic solvents containing at least one hydrophobic pharmaceutically active substance, water-insoluble organic polymeric material and amphiphilic organic polymeric material,

(b) treatment of the solution with supersonics,

(c) dialysis of the solution against H₂O

(d) separation of the resulting particles from the resulting aqueous solution.

26. A medicinal drug containing particles as defined in any one of claims 1 to 22 and, optionally, suitable additives and/or auxiliaries.

27. A method of using particles as defined in any one of claims 1 to 22 for the production of a medicinal drug for treatment of cancer, for treatment of infectious diseases and parasitosis, for treatment of diseases and symptoms caused by the central nervous system, for use in genetic therapy, or for genomic targeting.

1. A monomolecular solid particle for transportation of hydrophobic or hydrophobed active substances, containing
a) an unbranched or at most triple-branched molecular backbone of a polymer composed of monomers and having at least one bonding group (x) on each monomer, there being attached to each of the bonding groups, (x) via a covalent (x)-(x′) bond,
b) polycondensed molecular side chains composed of chain-forming monomers having in each case at least one bonding group (y) and at least one bonding group (x′) or composed of different chain-forming monomers, of which one monomer has at least two bonding groups (y) and a different monomer has at least two bonding groups (x′), or composed of different chain-forming monomers, of which one monomer has at least two bonding groups (y), a different monomer has at least two bonding groups (x′) and another monomer has at least one bonding group (y) and at least one bonding group (x′), where (x′) is a group capable of forming a covalent bond with (x) and also a covalent bond with (y), whilst at the end of each molecular side chain there is attached, via a (y)-(y′) covalent bond
c) a side-chain terminator having at least one bonding group (y′), no bonding group (y) and at least one free group (z) optionally provided with a protective group, characterized in that
the molar ratio of the monomers in the molecular side chains (b) to the side-chain terminators (c) is >>1,
group y≠group z and group x′≠group z,
x, x′, y and y′ are independently selected from the group comprising OH, SH, COOH and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide bond NH—C(O)—), SH/COOH (thioester bond —S—C(O)—), COOH/OH (ester bond —O—C(O)—), COOH/NH₂ (amide bond.—NH—C(O)—) or COOH/SH (thioester bond S—C(O)—) and
z is selected from the group comprising CH₃, OH, SH, COOH, NH₂, and the vinyl or epoxide group, optionally protected by a suitable protective group,

2. A particle as defined in claim 1, characterized in that it contains a non-covalently bonded, hydrophobic or hydrophobed pharmaceutically active substance.

3. A monomolecular solid particle for transportation of hydrophobic or hydrophobed active substances obtainable by a process, in which an unbranched or at most triple-branched polymer backbone composed of monomers having at least one bonding group (x) on each monomer is brought into contact with
a) chain-forming side-chain monomers each of which has at least one bonding group (y) and at least one bonding group (x′), where (x′) is a group capable of forming a a covalent bond both with (x) and with (y),
b) a mixture of chain-forming side-chain monomers, of which at least one monomer has at least two bonding groups (y) and, on at least one different monomer, at least two bonding groups (x′), where (x′) is a group which can form a covalent bond both with (x) and with (y), or
c) a mixture of chain-forming side-chain monomers, of which at least one monomer has at least two bonding groups (y), at least one different monomer has at least two bonding groups (x′) and at least one other monomer has at least one bonding group (y) and at least one bonding group (x′), where (x′) is a group which can form a covalent bond both with (x) and with (y),
and also at least one side-chain terminator having at least one bonding group (y′), no bonding group (y), and at least one free group (z) optionally provided with a protective group, where (y′) is a group which can form a covalent bond with (y),
under conditions permitting bonding between the monomers or monomer mixtures of the side chains and the polymer backbone and the chain terminators and also permitting polycondensation of the monomers or monomer mixtures in the side chains, characterized in that
the molar ratio of the monomers in the molecular side chains (b) to the side-chain terminators (c) is >>1,
group y≠group z and group x′≠group z,
the molar ratio of the monomers in the molecular backbone to the side-chain terminators (c) is approximately equimolar
x, x′, y, and y′ are independently selected from the group comprising OH, SH, COOH, or NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide linkage —NH—C(O)—), SH/COOH (thioester bond —S—C(O)—), COOH/OH (ester bond —O—C(O)—), COOH/NH₂ (amide linkage —NH—C(O)—) or COOH/SH (thioester bond S—C(O)—),
z is selected from the group comprising CH₃, OH, SH, COOH, NH₂, and the vinyl or epoxide group, optionally protected by a protective group, and
the side-chain monomers can be used in the form of pure monomers or derivatives such as inner anhydrides or lactones, provided they are still capable of forming chains with themselves and/or with other side-chain monomers.

4. A monomolecular solid particle for transportation of hydrophobic or hydrophobed active substances obtainable by a process, in which an unbranched or at most triple-branched polymer backbone composed of monomers having at least one bonding group (x) on each monomer is brought into contact with
polycondensed molecular side chains composed of monomers, of which each monomer has at least one bonding group (y) and at least one bonding group (x′), or composed of different monomers, of which one monomer has at least two bonding groups (y) and a different monomer has at least two bonding groups (x′), or composed of different monomers, of which one monomer has at least two bonding groups (y), a different monomer has at least two bonding groups (x′), and another monomer has at least one bonding group (y) and at least one bonding group (x′), where (x′) is a group which can form a covalent bond both with (x) and with (y), in which side-chain terminators are covalently bonded to the end of each of the molecular side chains via a (y)-(y′) bond, which terminators have at least one bonding group (y′), no bonding group (y) and at least one free group (z) optionally provided with a protective group, where (y′) is a group which can form a covalent bond with (y), under conditions which allow for linkage between the polycondensed molecular side chains and the polymer, characterized in that
the molar ratio of the monomers in the molecular side chains (b) to the side-chain terminators (c) is >>1,
group y≠group z and group x′≠group z,
the molar ratio of the monomers in the molecular backbone to the side-chain terminators (c) is approximately equimolar
x, x′, y, and y′ are independently selected from the group comprising OH, SH, COOH, and NH₂ with the proviso that x/x′, x/y, or y/y′ form corresponding bond pairs x/x′, x′/y, or y/y′ (with the corresponding bond) selected from the group comprising OH/COOH (ester bond —O—C(O)—), NH₂/COOH (amide linkage —NH—C(O)—), SH/COOH (thioester bond —S—C(O)—), COOH/OH (ester bond —O—C(O)—), COOH/NH₂ (amide linkage —NH—C(O)—) or COOH/SH (thioester bond S—C(O)—) and
z is selected from the group comprising CH₃, OH, SH, COOH, and NH₂, and also the vinyl or epoxide group optionally protected by a protective group.

5. A solid particle for transportation of substances as defined in claim 3 or claim 4, characterized in that contacting is carried out in an anhydrous organic solvent, preferably pyridine.

6. A solid particle for transportation of substances as defined in any one of claims 3 to 5, characterized in that contacting is carried out in the presence of thionyl chloride.

7. A particle as defined in any one of claims 3 to 6, characterized in that any protective groups present are eliminated.

8. A particle as defined in any one of claims 3 to 7, characterized in that a washing step, preferably with dichloromethane, is subsequently carried out.

9. A particle as defined in any one of claims 3 to 8, characterized in that the product is then dissolved, together with the hydrophobic or hydrophobed pharmaceutically active substance to be transported, in an anhydrous organic solvent, after which the solution is incubated for some time, preferably overnight, preferably at room temperature, and the solution is then saturated with water and the resulting water-saturated solution is dissolved in a larger volume of water, followed, optionally, by mechanical treatment and treatment for cleaning the particles, which are then isolated.

10. A particle as defined in claim 9, characterized in that, after the water-saturated solution has been dissolved in a larger volume of water, no mechanical treatment is carried out and/or the cleaning by dialysis, preferably against H₂O, is carried out.

11. A particle as defined in claim 9 or claim 10, characterized in that the anhydrous organic solvent dissolves in water in a ratio of solvent to water between 1:10 and 1:50, preferably between 1:20 and 1:40, and more preferably between 1:20 and 1:30, and/or is preferably selected from the group comprising:
dichloromethane or benzyl alcohol, preferably benzyl alcohol.

12. A particle as defined in any one of claims 1 to 11, characterized in that the polymer backbone is unbranched or branched not more than once, but is preferably unbranched.

13. A particle as defined in any one of claims 1 to 12, characterized in that
the monomers in the side chain each have not more than two groups (y) and not more than two groups (x′) and/or
group (y) in the monomers in the side chain is the same as group (x) in the polymer backbone and/or
group (x′) in the monomers in the side chain is the same as group (y′) in the side-chain terminator and/or
group (z) is selected from the “free groups” OH, SH, COOH, and NH₂, and also the vinyl or epoxide group optionally protected by a protective group,
the monomers in the side chain each have not more than from 2 to 10 carbon atoms, preferably from 2 to 6 carbon atoms, and more preferably from 2 to 4 carbon atoms and/or
the monomers in the side chain, which contain both group (y) and group (x′), have either only 1 group (y) and 1 or 2, preferably 1, groups (x′) or only 1 group (x′) and 1 or 2, preferably 1, groups (y) and/or
the monomers in the side chains are, except for the side-chain terminator, each identical to 1 group (y) and 1 group (x′), or the monomers in the side chains are, except for the side-chain terminator, identical to monotonously alternating monomers comprising a monomer containing two groups (x′) alternating with a monomer containing two groups (y).

14. A particle as defined in any one of claims 1 to 13, characterized in that the polymer backbone is selected from the group comprising
polyvinyl alcohol, polyacrylic acid, polyvinyl amine, polysaccharide, and polyamino acid,
preferably polyvinyl alcohol or polyacrylic acid, more preferably polyvinyl alcohol.

15. A particle as defined in any one of claims 1 to 14, characterized in that the monomers in the side chain are selected from the group comprising
hydroxycarboxylic acids, amino acids, combinations of diamines and dicarboxylic acids, combinations of diols and dicarboxylic acids, or derivatives thereof
preferably hydroxycarboxylic acids or a combination of diols and dicarboxylic acids, or derivatives thereof,
more preferably hydroxycarboxylic acids such as lactic acid, glycolic acid, tartaric acid, citric acid, or derivatives thereof.

16. A particle as defined in any one of claims 1 to 15, characterized in that the side-chain terminators are selected from the group comprising
unprotected amino acids, N-protected amino acids, COOH-protected amino acids, unprotected amino alcohols, N-protected amino alcohols, O-protected amino alcohols, unprotected thiols, O-protected thiols, S-protected thiols, unprotected thiolic acids, S-protected thiolic acids, COOH-protected thiolic acids, unprotected thioamines, S-protected thioamines, and N-protected thioamines, preferably
unprotected amino acids, N-protected amino acids, unprotected amino alcohols, N-protected amino alcohols, unprotected thiols, S-protected thiols, unprotected thiolic acids, and S-protected thiolic acids, more preferably
unprotected amino acids, such as alanine, N-protected amino acids, such as N-FMOC-9-alanine, unprotected thiolic acids, and S-protected thiolic acids.

17. A particle as defined in any one of claims 1 to 16, characterized in that the polymer backbone, the monomers in the side chain or derivatives thereof, and the side-chain terminators or derivatives thereof are selected from one of the following combinations:

| Comb. | Polymeric | Monomer(s) in side-chain | Side-chain terminator |
|---|---|---|---|
| No. | backbone | or derivative(s) thereof | or derivative thereof |
| 1 | Polyvinyl alcohol | Hydroxycarboxylic acid | Unprotected amino acid |
| 2 | Polyvinyl alcohol | Hydroxycarboxylic acid | N-protected amino acid |
| 3 | Polyvinyl alcohol | Hydroxycarboxylic acids | Unprotected thiolic acid |
| 4 | Polyvinyl alcohol | Hydroxycarboxylic acids | S-protected thiolic acid |
| 5 | Polyvinyl alcohol | Combination of diol and dicarboxylic acid | Unprotected amino acid |
| 6 | Polyvinyl alcohol | Combination of diol and dicarboxylic acid | N-protected amino acid |
| 7 | Polyvinyl alcohol | Combination of diol and dicarboxylic acid | Unprotected thiolic acid |
| 8 | Polyvinyl alcohol | Combination of diol and dicarboxylic acid | S-protected thiolic acid |
| 9 | Polyvinyl alcohol | Amino acid | Unprotected thiolic acid |
| 10 | Polyvinyl alcohol | Amino acid | S-protected thiolic acid |
| 11 | Polyvinyl alcohol | Combination of diamine and dicarboxylic acid | Unprotected thiolic acid |
| 12 | Polyvinyl alcohol | Combination of diamine and dicarboxylic acid | S-protected thiolic acid |
| 13 | Polyacrylic acid | Hydroxycarboxylic acid | Unprotected amino alcohol |
| 14 | Polyacrylic acid | Hydroxycarboxylic acid | N-protected amino alcohol |
| 15 | Polyacrylic acid | Hydroxycarboxylic acid | Unprotected thiol |
| 16 | Polyacrylic acid | Hydroxycarboxylic acid | S-protected thiol |
| 17 | Polyacrylic acid | Combination of diol and dicarboxylic acid | Unprotected amino alcohol |
| 18 | Polyacrylic acid | Combination of diol and dicarboxylic acid | N-protected amino alcohol |
| 19 | Polyacrylic acid | Combination of diol and dicarboxylic acid | Unprotected thiol |
| 20 | Polyacrylic acid | Combination of diol and dicarboxylic acid | S-protected thiol |
| 21 | Polyacrylic acid | Amino acid | Unprotected amino alcohol |
| 22 | Polyacrylic acid | Amino acid | O-protected amino alcohol |
| 23 | Polyacrylic acid | Amino acid | Unprotected thioamine |
| 24 | Polyacrylic acid | Amino acid | S-protected thioamine |
| 25 | Polyacrylic acid | Combination of diamine and dicarboxylic acid | Unprotected amino alcohol |
| 26 | Polyacrylic acid | Combination of diamine and dicarboxylic acid | O-protected amino alcohol |
| 27 | Polyacrylic acid | Combination of diamine and dicarboxylic acid | Unprotected thioamine |
| 28 | Polyacrylic acid | Combination of diamine and dicarboxylic acid | S-protected thioamine |
| 29 | Polyvinyl amine | Hydroxycarboxylic acids | Unprotected amino acid |
| 30 | Polyvinyl amine | Hydroxycarboxylic acids | N-protected amino acid |
| 31 | Polyvinyl amine | Hydroxycarboxylic acids | Unprotected thiolic acid |
| 32 | Polyvinyl amine | Hydroxycarboxylic acids | S-protected thiolic acid |
| 33 | Polyvinyl amine | Combination of diol and dicarboxylic acid | Unprotected amino acid |
| 34 | Polyvinyl amine | Combination of diol and dicarboxylic acid | N-protected amino acid |
| 35 | Polyvinyl amine | Combination of diol and dicarboxylic acid | Unprotected thiolic acid |
| 36 | Polyvinyl amine | Combination of diol and dicarboxylic acid | S-protected thiolic acid |
| 37 | Polyvinyl amine | Amino acid | Unprotected thiolic acid |
| 38 | Polyvinyl amine | Amino acid | S-protected thiolic acid |
| 39 | Polyvinyl amine | Combination of diamine and dicarboxylic acid | Unprotected thiolic acid |
| 40 | Polyvinyl amine | Combination of diamine and dicarboxylic acid | S-protected thiolic acid |
| 41 | Polysaccharide | Hydroxycarboxylic acid | Unprotected amino acid |
| 42 | Polysaccharide | Hydroxycarboxylic acid | N-protected amino acid |
| 43 | Polysaccharide | Hydroxycarboxylic acids | Unprotected thiolic acid |
| 44 | Polysaccharide | Hydroxycarboxylic acids | S-protected thiolic acid |
| 45 | Polysaccharide | Combination of diol and dicarboxylic acid | Unprotected amino acid |
| 46 | Polysaccharide | Combination of diol and dicarboxylic acid | N-protected amino acid |
| 47 | Polysaccharide | Combination of diol and dicarboxylic acid | Unprotected thiolic acid |
| 48 | Polysaccharide | Combination of diol and dicarboxylic acid | S-protected thiolic acid |
| 49 | Polysaccharide | Amino acid | Unprotected thiolic acid |
| 50 | Polysaccharide | Amino acid | S-protected thiolic acid |
| 51 | Polysaccharide | Combination of diamine and dicarboxylic acid | Unprotected thiolic acid |
| 52 | Polysaccharide | Combination of diamine and dicarboxylic acid | S-protected thiolic acid |
| 53 | Polycystein | Hydroxycarboxylic acids | Unprotected amino acid |
| 54 | Polycystein | Hydroxycarboxylic acids | N-protected amino acid |
| 55 | Polycystein | Hydroxycarboxylic acids | Unprotected thiolic acid |
| 56 | Polycystein | Hydroxycarboxylic acids | S-protected thiolic acid |
| 57 | Polycystein | Combination of diol and dicarboxylic acid | Unprotected amino acid |
| 58 | Polycystein | Combination of diol and dicarboxylic acid | N-protected amino acid |
| 59 | Polycystein | Combination of did and dicarboxylic acid | Unprotected thiolic acid |
| 60 | Polycystein | Combination of diol and dicarboxylic acid | S-protected thiolic acid |
| 61 | Polycystein | Amino acid | Unprotected thiolic acid |
| 62 | Polycystein | Amino acid | S-protected thiolic acid |
| 63 | Polycystein | Combination of diol and dicarboxylic acid | Unprotected thiolic acid |
| 64 | Polycystein | Combination of diol and dicarboxylic acid | S-protected thiolic acid |
| 65 | Polyserin | Hydroxycarboxylic acid | Unprotected amino acid |
| 66 | Polyserin | Hydroxycarboxylic acid | N-protected amino acid |
| 67 | Polyserin | Hydroxycarboxylic acids | Unprotected thiolic acid |
| 68 | Polyserin | Hydroxycarboxylic acids | S-protected thiolic acid |
| 69 | Polyserin | Combination of diol and dicarboxylic acid | Unprotected amino acid |
| 70 | Polyserin | Combination of diol and dicarboxylic acid | N-protected amino acid |
| 71 | Polyserin | Combination of diol and dicarboxylic acid | Unprotected thiolic acid |
| 72 | Polyserin | Combination of diol and dicarboxylic acid | S-protected thiolic acid |
| 73 | Polyserin | Amino acid | Unprotected thiolic acid |
| 74 | Polyserin | Amino acid | O-protected thiolic acid |
| 75 | Polyserin | Combination of diamine and dicarboxylic acid | Unprotected thiolic acid |
| 76 | Polyserin | Combination of diamine and dicarboxylic acid | S-protected thiolic acid |

preferably

| | | | |
|---|---|---|---|
| 81 | Polyvinyl alcohol | Glycolic acid | Unprotected amino acid |
| 82 | Polyvinyl alcohol | Glycolic acid | N-protected amino acid |
| 83 | Polyvinyl alcohol | Glycolic acid | β-alanine |
| 84 | Polyvinyl alcohol | Glycolic acid | N-FMOC-β-alanine |
| 85 | Polyvinyl alcohol | Tartaric acid | Unprotected amino acid |
| 86 | Polyvinyl alcohol | Tartaric acid | N-protected amino acid |
| 87 | Polyvinyl alcohol | Tartaric acid | β-alanine |
| 88 | Polyvinyl alcohol | Tartaric acid | N-FMOC-β-alanine |
| 89 | Polyvinyl alcohol | Citric acid | Unprotected amino acid |
| 90 | Polyvinyl alcohol | Citric acid | N-protected amino acid |
| 91 | Polyvinyl alcohol | Citric acid | β-alanine |
| 92 | Polyvinyl alcohol | Citric acid | N-FMOC-Walanine |
| 93 | Polyacrylic acid | Lactic acid | Unprotected amino acid |
| 94 | Polyacrylic acid | Lactic acid | N-protected amino acid |
| 95 | Polyacrylic acid | Lactic acid | Aminoethanol |
| 96 | Polyacrylic acid | Lactic acid | N-FMOC-colamine |
| 97 | Polyacrylic acid | Glycolic acid | Unprotected amino acid |
| 98 | Polyacrylic acid | Glycolic acid | N-protected amino acid |
| 99 | Polyacrylic acid | Glycolic acid | Aminoethanol |
| 100 | Polyacrylic acid | Glycolic acid | N-FMOC-colamine |
| 101 | Polyacrylic acid | Tartaric acid | Unprotected amino alcohol |
| 102 | Polyacrylic acid | Tartaric acid | N-protected amino alcohol |
| 103 | Polyacrylic acid | Tartaric acid | Aminoethanol |
| 104 | Polyacrylic acid | Tartaric acid | N-FMOC-colamine |
| 105 | Polyacrylic acid | Citric acid | Unprotected amino alcohol |
| 106 | Polyacrylic acid | Citric acid | N-protected amino alcohol |
| 107 | Polyacrylic acid | Citric acid | Aminoethanol |
| 108 | Polyacrylic acid | Citric acid | N-FMOC-colamine |

more preferably

| | | | |
|---|---|---|---|
| 79 | Polyvinyl alcohol | Lactic acid | β-alanine |
| 80 | Polyvinyl alcohol | Lactic acid | N-FMOC-β-alanine |
| 95 | Polyacrylic acid | Lactic acid | Aminoethanol |
| 96 | Polyacrylic acid | Lactic acid | N-FMOC-colamine |

18. A particle as defined in any one of claims 1 to 17, characterized in that the particle has a length of <5 μm, preferably <3 μm, and more preferably <2 μm and/or a thickness and width of <200 nm, preferably <75 nm, and more preferably <30 nm.

19. A particle as defined in any one of claims 1 to 18, characterized in that linker molecules containing a reactive group (z′), selected from groups capable of forming a covalent bond with one of the groups (z) selected from the “free groups” (z) selected from the group comprising OH, SH, COOH, and NH₂, and also the vinyl or epoxide group, preferably an amino-reactive or thiol-reactive group, more preferably an amino-reactive group, are covalently bonded to the particle, via (z′)-(z) bonds to groups (z) present on the surface of the particle and selected from the “free groups”.

20. A particle as defined in claim 19, characterized in that the linker molecules are bifunctional and contain, in addition to the reactive group (z′) binding to the particle as defined in any one of claims 1 to 18, also another reactive group (z″) at another end of the molecule, which reactive group (z″) is selected from the group comprising reactive groups capable of forming a covalent bond with one of the groups (z) selected from the “free groups” (z) selected from the group comprising OH, SH, COOH, and NH₂, and also the vinyl or epoxide group, preferably a thiol-reactive group, where z′≠z″.

21. A particle as defined in claim 19 or claim 20, characterized in that the linker molecules constitute a mixture of bifunctional molecules as defined in claim 20 and monofunctional molecules, which contain, in addition to the reactive group (z′) binding to the particle as defined in any one of claims 1 to 18, preferably an amino-reactive or thiol-reactive, preferably an amino-reactive, group, no functional group (z″) of different reactivity at any other end of the molecule, where z′≠z″.

22. A particle as defined in claim 21, characterized in that there are covalently bonded to the surface of the particle distinctly more, preferably at least 100% more, monofunctional linker molecules than bifunctional linker molecules.

23. A particle as defined in any one of claims 20 to 22, characterized in that bio-active macromolecules or “seeker”molecules, selected from the group comprising peptides, proteins; preferably antibodies, antibody fragments, or antibody derivatives showing target-binding properties such as “single-chain” antibodies; hormones, sugars, preferably glycosides; synthetic or natural receptor ligands; proteins or peptides containing a free cysteine group, and thiosugars, are coupled, will be coupled, or have been coupled, prior to surface modification, to the bifunctional linker molecules via a bond to the reactive group (z″).

24. A particle as defined in any one of claims 20 to 22, characterized in that bio-active macromolecules or “seeker” molecules, preferably antibodies, antibody fragments or antibody derivatives showing target-binding properties such as “single-chain” antibodies, particularly those containing a free cysteine group, are coupled, will be coupled, or have been coupled, prior to surface modification, to said bifunctional linker molecules.

25. A particle as defined in claim 20, characterized in that bio-active micromolecules or “seeker” molecules, preferably sugars, particularly thiosugars; and peptides and hormones, particularly those having a free cysteine group, are coupled, will be coupled, or have been coupled, prior to surface modification, to the bifunctional molecules of the coating via a bond to the reactive group (z″).

26. A particle as defined in any one of claims 23 to 25, characterized in that any reactive groups (z″) still free after attachment of the bio-active micromolecules or “seeker” molecules are saturated, preferably with cysteine.

27. A particle as defined in claim 19, characterized in that the linker molecules are monofunctional molecules which contain, in addition to the reactive group (z′) binding to the particle as defined in any one of claims 1 to 18, preferably an amino-reactive or thiol-reactive group, particularly an amino-reactive group, no reactive group (z″) of different reactivity at any other end of the molecule, where z′≠z″.

28. A particle as defined in any one of claims 19 to 27, characterized in that the linker molecules are polyglycolides, preferably polyethylene glycol derivatives, and more preferably NHS-ester-PEG or NHS-ester/vinylsulfone-PEG.

29. A particle as defined in any one of claims 1 to 28, characterized in that the active substance to be transported is a synthetic or natural active substance, a protein, peptide, lipid, sugar or nucleic acid or a low-molecular organic active substance or high-molecular organic active substance, for example, a hormone, a carcinostatic, an antibiotic agent, an antifungal agent, a parasiticide, a virustatic agent, an antihelminthicum, a substance showing cardiovascular activity; or a substance acting on the central nervous system, particularly an analgesic, antidepressant or antiepileptic.

30. A particle as defined in any one of claims 1 to 18, characterized in that it is directly coupled or is coupled via a linker, preferably via bifunctional polyethylene glycol molecules therein, to a “seeker” molecule selected from the group comprising:
peptides, proteins; preferably antibodies, antibody fragments, or antibody derivatives showing target-binding properties such as “single-chain” antibodies; hormones, sugars, preferably glycosides; synthetic or natural receptor ligands; proteins or peptides having a free cysteine group, or thiosugars.

31. A process for the synthesis of a particle as defined in claim 1 or claim 2, characterized in that an unbranched or at most triple-branched polymer backbone (a) composed of monomers each having at least one bonding group (x) on each monomer is brought into contact with
side-chain monomers (b) each containing at least one bonding group (y) and at least one bonding group (x′), where (x′) is a group which can form a covalent bond both with (x) and with (y),
a mixture of side-chain monomers (b), of which at least one monomer has at least two bonding groups (y) and at least one different monomer has at least two bonding groups (x′), in which (x′) is a group capable of forming a covalent bond both with (x) and with (y), or
a mixture of side-chain monomers (b), of which at least one monomer has at least two bonding groups (y), at least one different monomer has at least two bonding groups (x′) and at least one other monomer has at least one bonding group (y) and at least one bonding group (x′), where (x′) is a group which can form a covalent bond both with (x) and with (y),
and also at least one side-chain terminator (c) having at least one bonding group (y′), no bonding group (y), and at least one free group (z) optionally provided with a protective group, characterized in that
the molar ratio of the monomers in the molecular side chains (b) to the side-chain terminators (c) is >>1,
group y≠group z and group x′≠group z,
the molar ratio of the monomers in the molecular backbone (a) to the side-chain terminators (c) is approximately equimolar
x, x′, y, and y′ are independently selected from the group comprising OH, SH, COOH, and NH₂ with the proviso that x/x′, x′/y, or y/y′ form corresponding bond pairs x/x′, x′/y, or y/y′ (with the corresponding bond) selected from the group comprising OH/COOH (ester bond —O—C(O)—), NH₂/COOH (amide linkage —NH—C(O)—), SH/COOH (thioester bond —S—C(O)—), COOH/OH (ester bond —O—C(O)—), COOH/NH₂ (amide linkage —NH—C(O)—) or COOH/SH (thioester bond —S—C(O)—),
z is selected from the group comprising CH₃, OH, SH, COOH, NH₂, and the vinyl or epoxide group, optionally protected by a protective group, and
the side-chain monomers (b) can be used as pure monomers or derivatives such as inner anhydrides or lactones, provided they are still capable of forming chains with themselves and/or with other side-chain monomers,
under conditions permitting bonding between the monomers or monomer mixtures in the side chain and the polymer backbone and also the side-chain terminators, and also permitting polycondensation of the monomers or monomer mixtures in the side chain,
optionally in an anhydrous organic solvent, preferably pyridine, optionally in the presence of thionyl chloride and is then optionally cleaned, preferably by dialysis against H₂O, and optionally isolated,
and the particles are then optionally dissolved, together with the hydrophobic or hydrophobed active substance to be transported, in an anhydrous organic solvent, and the solution is then incubated for a period of time, preferably overnight, and preferably at room temperature, after which the solution is saturated with water and the water-saturated solution is then dissolved in a larger volume of water, followed, optionally, by mechanical treatment and, optionally, cleaning of the particles, preferably by dialysis against H₂O, followed by isolation of the particles.

32. A process for the production of a particle as defined in any one of claims 19 to 29, characterized in that a particle as defined in any one of claims 1 to 18 containing a group (z) selected from the “free groups” is brought into contact with a linker molecule containing a reactive group (z′) capable of forming a covalent bond with the group (z), under conditions suitable for the formation of this covalent bond, after which the particle is optionally cleaned, preferably by dialysis against H₂O, and isolated.

33. A process for the production of a particle as defined in any one of claims 23 to 25, characterized in that, following the process as defined in claim 32, the particle produced thereby having a free reactive group (z″) on bifunctional linker molecules is brought into contact with bio-active macromolecules or “seeker” molecules as defined in any one of claims 23 to 25 under conditions suitable for the formation of a bond between group (z″) and the bio-active macromolecules or “seeker” molecules, after which the particle is optionally cleaned, preferably by dialysis against H₂O, and isolated.

34. A medicinal drug containing particles as defined in any one of claims 1 to 30 and, optionally, suitable additives and/or auxiliaries.

35. A diagnostic reagent containing particles as defined in any one of claims 1 to 30 and, optionally, suitable additives and/or auxiliaries.

36. A method of using particles as defined in any one of claims 1 to 30 for the production of a medicinal drug for treatment of cancer, for treatment of infectious and parasitic diseases, for treatment of diseases and symptoms caused by the central nervous system, for use in genetic therapy, or for genomic targeting.

37. A solid particles for transportation of hydrophobic or hydrophobed pharmaceutically active substances, synthesized by a process comprising the following steps:
(a) preparation of a solution in an organic solvent or a mixture of organic solvents containing at least one hydrophobic or hydrophobed pharmaceutically active substance, water-insoluble organic polymeric material and amphiphilic organic polymeric material, and optionally supplementary substances,
(b) treatment of the solution with supersonics,
(c) dialysis of the solution against H₂O
(d) separation of the resulting particles from the resulting aqueous solution.

38. A process for the production of a particle as defined in claim 37, characterized by the following steps:
(a) preparation of a solution in an organic solvent or in a mixture of organic solvents containing at least one hydrophobic pharmaceutically active substance, water-insoluble organic polymeric material and amphiphilic organic polymeric material,
(b) treatment of the solution with supersonics,
(c) dialysis of the solution against H₂O,
(d) separation of the resulting particles from the resulting aqueous solutiom.
